(19) United States
(12) Patent Application Publication
(10) Pub. No.: US 2022/0117973 A1
(43) Pub. Date: Apr. 21, 2022

(54) THE ATR KINASE INHIBITOR BAY1895344 FOR USE IN THE TREATMENT OF A HYPER-PROLIFERATIVE DISEASE

(71) Applicant: Bayer Aktiengesellschaft, Leverkusen (DE)

(72) Inventors: Dennis KRICKAU, Berlin (DE); Eleni LAGKADINOU, Geneva (CH); Antje Margret WENGNER, Berlin (DE); Michael KRAUSE, Berlin (DE); Peter SERNO, Bergisch Gladbach (DE); Michaela BAIRLEIN, Wuppertal (DE); Gary WILKINSON, Berlin (DE); Siobhan WATTERS, Belfast (GB); Pardis ASSI, Lisburn (GB)

(73) Assignee: Bayer Aktiengesellschaft, Leverkusen (DE)

(21) Appl. No.: 17/310,483

(22) PCT Filed: Feb. 6, 2020
(86) PCT No.: PCT/EP2020/052971

§ 371 Date: Aug. 5, 2021

(30) Foreign Application Priority Data
Feb. 11, 2019 (EP) 19156399.8

(51) Int. Cl. A61K 31/5377; A61K 9/20; A61P 35/00

(52) U.S. Cl. CPC A61K 31/5377; A61K 9/2009; A61P 35/00; A61K 9/2054; A61K 9/2018

(57) ABSTRACT

The present invention relates to the ATR kinase inhibitor, 2-[(3R)-3-methylmorpholin-4-yl]-4-(1-methyl-1H-pyrazol-5-yl)-8-(1H-pyrazol-5-yl)-1,7-naphthyridine, for use in the treatment of a hyper-proliferative disease, characterized in that it is administered in an amount of from 10 mg to 160 mg per day, particularly in an amount of 60 to 160 mg per day. The present invention also relates to a pharmaceutical composition comprising 2-[(3R)-3-methylmorpholin-4-yl]-4-(1-methyl-1H-pyrazol-5-yl)-8-(1H-pyrazol-5-yl)-1,7-naphthyridine in an amount of from 5 mg to 80 mg and at least one pharmaceutically acceptable excipient. The present invention also relates to a process for manufacturing said pharmaceutical composition.

The present invention relates to the ATR kinase inhibitor, 2-[(3R)-3-methylmorpholin-4-yl]-4-(1-methyl-1H-pyrazol-5-yl)-8-(1H-pyrazol-5-yl)-1,7-naphthyridine, for use in the treatment of a hyper-proliferative disease, characterized in that it is administered in an amount of from 10 mg to 160 mg per day, particularly in an amount of 60 mg to 160 mg per day. The present invention also relates to a pharmaceutical composition comprising 2-[(3R)-3-methylmorpholin-4-yl]-4-(1-methyl-1H-pyrazol-5-yl)-8-(1H-pyrazol-5-yl)-1,7-naphthyridine in an amount of from 5 to 80 mg and at least one pharmaceutically acceptable excipient. The present invention also relates to a process for manufacturing said pharmaceutical composition.

### BACKGROUND OF THE INVENTION

2-[(3R)-3-methylmorpholin-4-yl]-4-(1-methyl-1H-pyrazol-5-yl)-8-(1H-pyrazol-5-yl)-1,7-naphthyridine is a compound of formula (I)

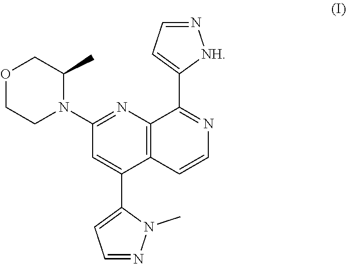 [SMILES: C[C@@H]1COCCN1c1cc(-c2ccnn2C)c2ccnc(-c3ccn[nH]3)c2n1]

It is also known as BAY1895344 and is an ATR kinase inhibitor, which can be used for the treatment of hyper-proliferative diseases. The synthesis of this compound is described in International Patent Publication WO2016/020320, particularly in its Example 111.

According to the Clinical Study Protocol and based on data obtained from in-vivo experiments after administration of BAY1895344 to mice, rats, and dogs, and from in-vitro data for plasma binding protein and blood/plasma partitioning, an efficacious dose in humans of more than 300 mg BID, 3 days on/4 days off, which means a dose of more than 600 mg per day, was estimated when starting clinical trials with BAY1895344.

One object of the invention is to identify one or more daily doses as well as dosing schedules of the compound of formula (I), which is/are suitable for the treatment of one or more hyper-proliferative disease(s). Another object of the invention is to identify pharmaceutical compositions comprising the compound of formula (I), which can be used for such treatment.

Despite the progress described in the art with regard to kinase inhibitors, there remains a need for improved pharmaceutical compositions for the treatment of hyper-proliferative diseases, in particular for pharmaceutical compositions, which are suitable for oral administration to increase the patient's compliance.

The release-profile of a pharmaceutical composition has to translate into a plasma level of the active ingredient which is sufficient for an effective therapy. Since the physico-chemical properties of active ingredients largely differ from each other, the types and amounts of the excipients to formulate a pharmaceutical composition, which is actually suitable to achieve the required plasma levels for a certain active ingredient, are generally not predictable.

Besides its dissolution properties, the chemical and mechanical stability of the pharmaceutical composition are of importance.

Finally, it is important that the process for manufacturing allows large-scale production of the pharmaceutical composition with high product quality.

The development of a reproducible large scale (≥2 kg) process for the GMP manufacture of a pharmaceutical composition comprising the compound of formula (I) turned out to be difficult. During upscaling of the GMP manufacture to 2 kg scale of pharmaceutical compositions comprising the compound of formula (I) different problems were encountered, such as flow and compaction issues, particularly during tableting (for details see Experimental Section below).

Another objective of the present invention therefore is to provide a process for the production of a pharmaceutical composition comprising the compound of formula (I), which allows production of said pharmaceutical composition, particularly at a scale of ≥2 kg.

### DESCRIPTION OF THE INVENTION

Compound for Use

In accordance with a first aspect, the present invention relates to 2-[(3R)-3-methylmorpholin-4-yl]-4-(1-methyl-1H-pyrazol-5-yl)-8-(1H-pyrazol-5-yl)-1,7-naphthyridine, or a tautomer, a solvate, a pharmaceutically acceptable salt thereof for use in the treatment of a hyper-proliferative disease, characterized in that it is administered in an amount of from 10 mg to 160 mg per day, particularly in an amount of from 60 mg to 160 mg per day, preferably in an amount of from 80 mg to 160 mg per day.

The compound of formula (I) can be administered once daily (QD) or twice daily (BID). Preferably it is administered twice daily (BID), e.g. to achieve the daily dose of from 10 mg to 160 mg, particularly in an amount of from 60 mg to 160 mg per day, preferably in an amount of from 80 mg to 160 mg per day.

Very surprisingly, the efficacious dose in humans of between 40 mg and 80 mg BID turned out to be more than 3 times (80 mg BID) and up to more than 7 times (40 mg BID) lower than the estimated efficacious dose of more than 300 mg BID, 3 days on/4 days off, which has been assumed when starting clinical trials with the compound of formula (I). This very significant difference between the estimated and the actual dose could not have been predicted and is very surprising in view of the preclinical pharmacokinetic data.

“2-[(3R)-3-methylmorpholin-4-yl]-4-(1-methyl-1H-pyrazol-5-yl)-8-(1H-pyrazol-5-yl)-1,7-naphthyridine” is the compound of formula (I)

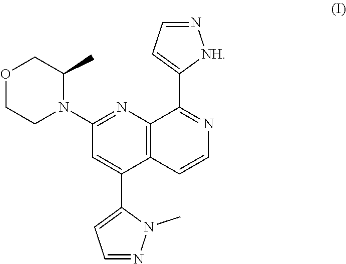 [SMILES: C[C@@H]1COCCN1c1cc(-c2ccnn2C)c2ccnc(-c3ccn[nH]3)c2n1]

The compound of formula (I) may exist as a tautomer of formula (Ia)

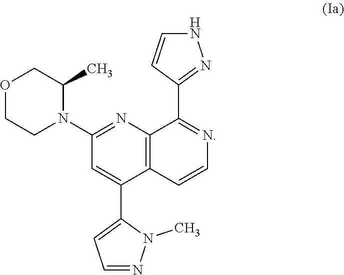 [SMILES: C[C@@H]1COCCN1c1cc(-c2ccnn2C)c2ccnc(-c3cc[nH]n3)c2n1]

The compound of formula (I) may exist in different solid forms, which are described in International Patent Publication WO2018/153970. Preferred are polymorphic forms, which are called “Form A” and “Form B”. Most preferred is “Form B”.

Methods for preparing different solid forms of the compound of formula (I), such as for example Form A and Form B, are described in International Patent Publication WO2018/153970.

Methods for preparing preferred “Form B” are described in Example 111 of International Patent Publication WO2016/020320A1 and in International Patent Publication WO2018/153970.

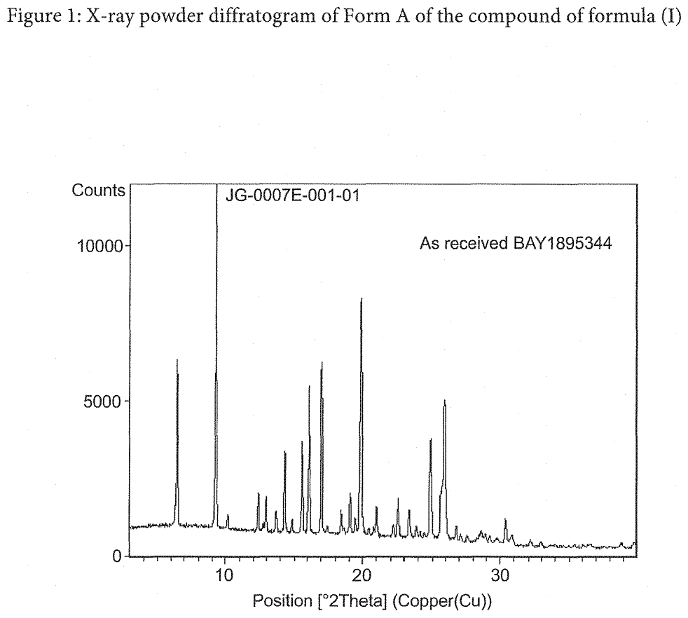

In accordance with an embodiment of the first aspect of the invention (the “compound for use”) Form A of the compound of formula (I) is administered. The X-ray powder diffractogram of Form A of the compound of formula (I) is shown in FIG. 1.

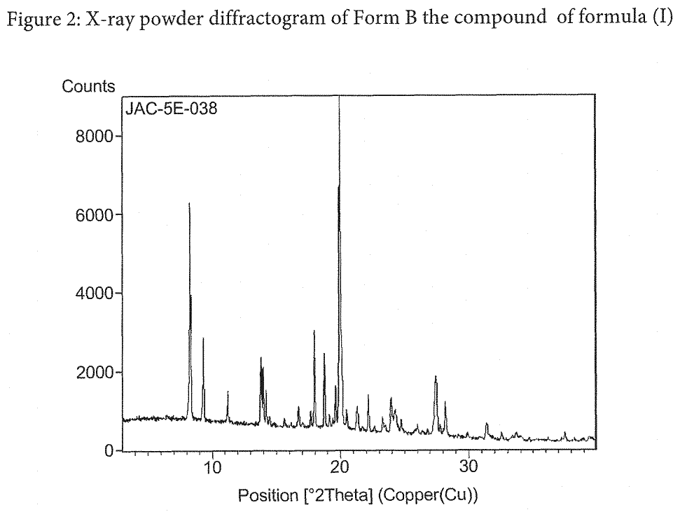

In accordance with a preferred embodiment of the first aspect of the invention Form B of the compound of formula (I) is administered. The X-ray powder diffractogram of Form B of the compound of formula (I) is shown in FIG. 2.

The compound of formula (I) may exist as a solvate, particularly as a hydrate.

Further, the compound of formula (I) can exist in the form of a salt. Said salt may be any salt, either an organic or inorganic addition salt, preferably any pharmaceutically acceptable salt, customarily used in pharmacy.

The term “pharmaceutically acceptable salt” refers to a relatively non-toxic, inorganic or organic acid addition salt of the compound of formula (I). For example, see S. M. Berge, et al. “Pharmaceutical Salts,” J. Pharm. Sci. 1977, 66, 1-19. A suitable pharmaceutically acceptable salt of the compound of formula (I). may be, for example, an acid-addition salt of a compound of formula (I), such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethanesulfonic, benzenesulfonic, para-toluenesulfonic, methansulfonic, 2-naphthalenesulfonic, naphthalenedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example. Preferred is an acid-addition salt of the compound of formula (I) with hydrochloric acid.

The compound of formula (I) or its tautomer, solvate (e.g. hydrate), pharmaceutically acceptable salt or any of the solid forms of the compound of formula (I) can be used in micronized form. Micronization can be achieved by standard milling methods.

The term “hyper-proliferative disease” includes, but is not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases.

The term “hyper-proliferative disease” also includes lymphomas, sarcomas, and leukemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalamic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

These diseases have been well characterized in humans, but also exist with a similar etiology in other mammals, and can be treated by administering a pharmaceutical composition of the present invention.

The hyper-proliferative disease can be characterized by one or more defect(s) in the DNA damage response (DDR) machinery. Particularly, the hyper-proliferative disease can be characterized by one or more biomarker(s), which were described in International Patent Publication WO2018/153968, which is herein incorporated by reference. Particularly, the hyper-proliferative disease can be characterized by one or more deleterious mutation(s) in one or more gene(s) and/or protein(s) specifically described in WO2018/153968. Preferably, the hyper-proliferative disease is characterized by one or more deleterious mutation(s) in ATM gene and/or BRCA1 gene and/or ATM protein and/or BRCA1 protein.

Further, the hyper-proliferative disease can be characterized by one or more deleterious mutation(s) in ATM gene and/or BRCA1 gene and/or BRCA2 gene and/or PALB2 gene and/or ARID1A gene and/or by a loss of ATM protein.

Preferably, the hyper-proliferative disease is characterized by one or more deleterious mutation(s) in ATM gene and/or BRCA1 gene and/or BRCA2 gene and/or by a loss of ATM protein.

Preferably, the hyper-proliferative disease is characterized by one or more deleterious mutation(s) in ATM gene.

Preferably, the hyper-proliferative disease is characterized by a loss of ATM protein.

Preferably, the hyper-proliferative disease is characterized by one or more deleterious mutation(s) in ATM gene and by a loss of ATM protein.

Preferably, the hyper-proliferative disease is characterized by one or more deleterious mutation(s) in BRCA1 gene.

Preferably, the hyper-proliferative disease is characterized by one or more deleterious mutation(s) in BRCA2 gene.

Further, the hyper-proliferative disease can be characterized by one or more deleterious mutation(s) in PALB2 gene.

Further, the hyper-proliferative disease can be characterized by one or more deleterious mutation(s) in ARID1A gene.

The term “deleterious mutation” as used herein means a mutation of a gene which has a deleterious effect on the function of said gene or on the function of its corresponding RNA or its corresponding protein.

For example, the deleterious mutation of the gene may result in a reduced gene expression level of said gene, or a reduced amount of the protein, particularly a loss of the protein, or in a reduced activity of the protein corresponding to said gene, or it may result in a nonfunctional gene/protein (“loss-of-function”) compared to the respective wildtype gene/protein.

Examples of a deleterious mutation include but are not limited to the following: The deleterious mutation can be a nonsense mutation, which is a point mutation in the respective gene, resulting in a premature stop codon, or a nonsense codon in the transcribed mRNA, and in a truncated, incomplete, and nonfunctional protein corresponding to the respective gene.

The deleterious mutation can be a missense mutation, which is a point mutation in the respective gene, resulting in the production either of a nonfunctional protein (complete loss of function) or in a protein with partial loss of function compared to the respective wildtype protein.

The deleterious mutation can also result in a frameshift mutation, which is a genetic mutation in the respective gene caused by insertions or deletions of one or more nucleotides in such gene, wherein the number of nucleotides is not divisible by three, and resulting in a (sometimes truncated) nonfunctional protein corresponding to the respective gene.

The deleterious mutation can also be a large rearrangement mutation, for example a deletion of one or more exons disrupting the reading frame or a critical functional domain of the corresponding protein. Another example for a large rearrangement mutation is a duplication of one or more non-terminal exons disrupting the reading frame or a critical functional domain of the corresponding protein.

The deleterious mutation can also be a splice site mutation, which is a genetic mutation that inserts, deletes or changes a number of nucleotides in the specific site at which splicing takes place during the processing of precursor messenger RNA into mature messenger RNA. Splice site consensus sequences that drive exon recognition are located at the very termini of introns. The deletion of the splicing site results in one or more introns remaining in mature mRNA thereby resulting in the production of a nonfunctional protein corresponding to the respective gene.

The deleterious mutation can also be a copy number variant (CNV), particularly a decrease of the gene copy number (e.g. a homozygous or heterozygous deletion) compared to the normal gene copy number of the respective gene.

For example, the deleterious mutation(s) in ATM gene and/or ATM protein may result in a loss of ATM protein and/or in a loss of ATM function. For example, the deleterious mutation(s) in BRCA1 gene and/or BRCA1 protein may result in a loss of BRCA1 protein and/or in a loss of BRCA1 function.

Methods for the determination of one or more deleterious mutation(s), for example in ATM or BRCA1 or BRCA2 or PALB2 or ARID1A gene and/or in ATM or BRCA1 or BRCA2 or PALB2 or ARID1A protein, are known to the person skilled in the art and are described, for example, in WO2018/153968, which is herein incorporated by reference.

In accordance with an embodiment of the first aspect the compound of formula (I) is administered in an amount of from 10 mg to 160 mg, or in an amount of from 20 mg to 120 mg, or in an amount of from 60 mg to 160 mg, or in an amount of from 20 mg to 80 mg per day, preferably in an amount of from 80 mg to 160 mg per day.

In accordance with an embodiment of the first aspect the compound of formula (I) is administered in an amount of 160 mg per day. The daily amount can be administered once daily (QD) or twice daily (BID). Preferably it is administered twice daily (BID), which means, that 80 mg are administered to the patient two times per day, for example, a first dose of 80 mg in the morning and a second dose of 80 mg in the evening Most preferably, the compound of formula (I) is administered twice daily (BID) every 12±1 hours.

In accordance with an embodiment of the first aspect the compound of formula (I) is administered in an amount of 80 mg (BID) and the dosing schedule is 1 day on/6 days off.

In accordance with an embodiment of the first aspect the compound of formula (I) is administered in an amount of 80 mg (BID) and the dosing schedule is 2 days on/5 days off.

In accordance with an embodiment of the first aspect the compound of formula (I) is administered in an amount of 80 mg (BID) and the dosing schedule is 3 days on/4 days off.

In accordance with an embodiment of the first aspect the compound of formula (I) is administered in an amount of 80 mg (BID) and the dosing schedule is 3 days on/11 days off. In other words, in this embodiment the dosing schedule is 3 days on/4 days off for one week, followed by a one week break without treatment.

In accordance with a preferred embodiment of the first aspect the compound of formula (I) is administered in an amount of 80 mg (BID) and the dosing schedule is 3 days on/11 days off.

In accordance with an embodiment of the first aspect the compound of formula (I) is administered in an amount of 80 mg (BID), the dosing schedule is 3 days on/4 days off for two weeks, followed by a one week break without treatment.

In an embodiment of the first aspect the compound of formula (I) is administered in an amount of 80 mg (BID), the dosing schedule is 3 days on/4 days off for three weeks, followed by a one week break without treatment.

In accordance with an embodiment of the first aspect the compound of formula (I) is administered in an amount of 140 mg per day. The daily amount can be administered once daily (QD) or twice daily (BID). Preferably it is administered twice daily (BID), which means, that 70 mg are administered to the patient two times per day, for example, a first dose of 70 mg in the morning and a second dose of 70 mg in the evening Most preferably, the compound of formula (I) is administered twice daily (BID) every 12±1 hours.

In accordance with an embodiment of the first aspect the compound of formula (I) is administered in an amount of 70 mg (BID) and the dosing schedule is 1 day on/6 days off.

In accordance with an embodiment of the first aspect the compound of formula (I) is administered in an amount of 70 mg (BID) and the dosing schedule is 2 days on/5 days off.

In accordance with an embodiment of the first aspect the compound of formula (I) is administered in an amount of 70 mg (BID) and the dosing schedule is 3 days on/4 days off.

In accordance with an embodiment of the first aspect the compound of formula (I) is administered in an amount of 70 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 70 mg (BID), the dosing schedule is 3 days on/4 days off for two weeks, followed by a one week break without treatment.

In an embodiment of the first aspect the compound of formula (I) is administered in an amount of 70 mg (BID), the dosing schedule is 3 days on/4 days off for three weeks, followed by a one week break without treatment.

In accordance with an embodiment of the first aspect the compound of formula (I) is administered in an amount of 120 mg per day. The daily amount can be administered once daily (QD) or twice daily (BID). Preferably it is administered twice daily (BID), which means, that 60 mg are administered to the patient two times per day, for example, a first dose of 60 mg in the morning and a second dose of 60 mg in the evening Most preferably, the compound of formula (I) is administered twice daily (BID) every 12±1 hours.

In accordance with an embodiment of the first aspect the compound of formula (I) is administered in an amount of 60 mg (BID) and the dosing schedule is 1 day on/6 days off.

In accordance with an embodiment of the first aspect the compound of formula (I) is administered in an amount of 60 mg (BID) and the dosing schedule is 2 days on/5 days off.

In accordance with an embodiment of the first aspect the compound of formula (I) is administered in an amount of 60 mg (BID) and the dosing schedule is 3 days on/4 days off.

In accordance with an embodiment of the first aspect the compound of formula (I) is administered in an amount of 60 mg (BID) and the dosing schedule is 3 days on/11 days off. In other words, in this embodiment the dosing schedule is 3 days on/4 days off for one week, followed by a one week break without treatment.

In accordance with a preferred embodiment of the first aspect the compound of formula (I) is administered in an amount of 60 mg (BID) and the dosing schedule is 3 days on/11 days off.

In accordance with an embodiment of the first aspect the compound of formula (I) is administered in an amount of 60 mg (BID), the dosing schedule is 3 days on/4 days off for two weeks, followed by a one week break without treatment.

In an embodiment of the first aspect the compound of formula (I) is administered in an amount of 60 mg (BID), the dosing schedule is 3 days on/4 days off for three weeks, followed by a one week break without treatment.

In accordance with an embodiment of the first aspect the compound of formula (I) is administered in an amount of 110 mg per day. The daily amount can be administered once daily (QD) or twice daily (BID). Preferably it is administered twice daily (BID), which means, that 55 mg are administered to the patient two times per day, for example, a first dose of 55 mg in the morning and a second dose of 55 mg in the evening Most preferably, the compound of formula (I) is administered twice daily (BID) every 12±1 hours.

In accordance with an embodiment of the first aspect the compound of formula (I) is administered in an amount of 55 mg (BID) and the dosing schedule is 2 days on/5 days off.

In accordance with an embodiment of the first aspect the compound of formula (I) is administered in an amount of 55 mg (BID) and the dosing schedule is 3 days on/4 days off.

In accordance with an embodiment of the first aspect the compound of formula (I) is administered in an amount of 55 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 55 mg (BID), the dosing schedule is 3 days on/4 days off for two weeks, followed by a one week break without treatment.

In an embodiment of the first aspect the compound of formula (I) is administered in an amount of 55 mg (BID), the dosing schedule is 3 days on/4 days off for three weeks, followed by a one week break without treatment.

In accordance with an embodiment of the first aspect the compound of formula (I) is administered in an amount of 100 mg per day. The daily amount can be administered once daily (QD) or twice daily (BID). Preferably it is administered twice daily (BID), which means, that 50 mg are administered to the patient two times per day, for example, a first dose of 50 mg in the morning and a second dose of 50 mg in the evening Most preferably, the compound of formula (I) is administered twice daily (BID) every 12±1 hours.

In accordance with an embodiment of the first aspect the compound of formula (I) is administered in an amount of 50 mg (BID) and the dosing schedule is 2 days on/5 days off.

In accordance with an embodiment of the first aspect the compound of formula (I) is administered in an amount of 50 mg (BID) and the dosing schedule is 3 days on/4 days off.

In accordance with an embodiment of the first aspect the compound of formula (I) is administered in an amount of 50 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 50 mg (BID), the dosing schedule is 3 days on/4 days off for two weeks, followed by a one week break without treatment.

In an embodiment of the first aspect the compound of formula (I) is administered in an amount of 50 mg (BID), the dosing schedule is 3 days on/4 days off for three weeks, followed by a one week break without treatment.

In accordance with an embodiment of the first aspect the compound of formula (I) is administered in an amount of 50 mg (BID) and the dosing schedule is 4 days on/3 days off.

In accordance with an embodiment of the first aspect the compound of formula (I) is administered in an amount of 90 mg per day. The daily amount can be administered once daily (QD) or twice daily (BID). Preferably it is administered twice daily (BID), which means, that 45 mg are administered to the patient two times per day, for example, a first dose of 45 mg in the morning and a second dose of 45 mg in the evening Most preferably, the compound of formula (I) is administered twice daily (BID) every 12±1 hours.

In accordance with an embodiment of the first aspect the compound of formula (I) is administered in an amount of 45 mg (BID) and the dosing schedule is 2 days on/5 days off.

In accordance with an embodiment of the first aspect the compound of formula (I) is administered in an amount of 45 mg (BID) and the dosing schedule is 3 days on/4 days off.

In accordance with an embodiment of the first aspect the compound of formula (I) is administered in an amount of 45 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 45 mg (BID), the dosing schedule is 3 days on/4 days off for two weeks, followed by a one week break without treatment.

In an embodiment of the first aspect the compound of formula (I) is administered in an amount of 45 mg (BID), the dosing schedule is 3 days on/4 days off for three weeks, followed by a one week break without treatment.

In accordance with an embodiment of the first aspect the compound of formula (I) is administered in an amount of 45 mg (BID) and the dosing schedule is 4 days on/3 days off.

In accordance with an embodiment of the first aspect the compound of formula (I) is administered in an amount of 45 mg (BID) and the dosing schedule is 5 days on/2 days off.

In accordance with an embodiment of the first aspect the compound of formula (I) is administered in an amount of 80 mg per day. The daily amount can be administered once daily (QD) or twice daily (BID). Preferably it is administered twice daily (BID), which means, that 40 mg are administered to the patient two times per day, for example, a first dose of 40 mg in the morning and a second dose of 40 mg in the evening Most preferably, the compound of formula (I) is administered twice daily (BID) every 12±1 hours.

In accordance with an embodiment of the first aspect the compound of formula (I) is administered in an amount of 40 mg (BID) and the dosing schedule is 2 days on/5 days off.

In accordance with an embodiment of the first aspect the compound of formula (I) is administered in an amount of 40 mg (BID) and the dosing schedule is 3 days on/4 days off.

In accordance with a preferred embodiment of the first aspect the compound of formula (I) is administered in an amount of 40 mg (BID) and the dosing schedule is 3 days on/4 days off.

In accordance with an embodiment of the first aspect the compound of formula (I) is administered in an amount of 40 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 40 mg (BID), the dosing schedule is 3 days on/4 days off for two weeks, followed by a one week break without treatment.

In an embodiment of the first aspect the compound of formula (I) is administered in an amount of 40 mg (BID), the dosing schedule is 3 days on/4 days off for three weeks, followed by a one week break without treatment.

In accordance with an embodiment of the first aspect the compound of formula (I) is administered in an amount of 40 mg (BID) and the dosing schedule is 4 days on/3 days off.

In accordance with an embodiment of the first aspect the compound of formula (I) is administered in an amount of 40 mg (BID) and the dosing schedule is 5 days on/2 days off.

In accordance with an embodiment of the first aspect the compound of formula (I) is administered in an amount of 70 mg per day. The daily amount can be administered once daily (QD) or twice daily (BID). Preferably it is administered twice daily (BID), which means, that 35 mg are administered to the patient two times per day, for example, a first dose of 35 mg in the morning and a second dose of 35 mg in the evening Most preferably, the compound of formula (I) is administered twice daily (BID) every 12±1 hours.

In accordance with an embodiment of the first aspect the compound of formula (I) is administered in an amount of 35 mg (BID) and the dosing schedule is 3 days on/4 days off.

In accordance with an embodiment of the first aspect the compound of formula (I) is administered in an amount of 35 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 35 mg (BID), the dosing schedule is 3 days on/4 days off for two weeks, followed by a one week break without treatment.

In an embodiment of the first aspect the compound of formula (I) is administered in an amount of 35 mg (BID), the dosing schedule is 3 days on/4 days off for three weeks, followed by a one week break without treatment.

In accordance with an embodiment of the first aspect the compound of formula (I) is administered in an amount of 35 mg (BID) and the dosing schedule is 4 days on/3 days off.

In accordance with an embodiment of the first aspect the compound of formula (I) is administered in an amount of 35 mg (BID) and the dosing schedule is 5 days on/2 days off.

In accordance with an embodiment of the first aspect the compound of formula (I) is administered in an amount of 60 mg per day. The daily amount can be administered once daily (QD) or twice daily (BID). Preferably it is administered twice daily (BID), which means, that 30 mg are administered to the patient two times per day, for example, a first dose of 30 mg in the morning and a second dose of 30 mg in the evening Most preferably, the compound of formula (I) is administered twice daily (BID) every 12±1 hours.

In accordance with an embodiment of the first aspect the compound of formula (I) is administered in an amount of 30 mg (BID) and the dosing schedule is 3 days on/4 days off.

In accordance with an embodiment of the first aspect the compound of formula (I) is administered in an amount of 30 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 30 mg (BID), the dosing schedule is 3 days on/4 days off for two weeks, followed by a one week break without treatment.

In an embodiment of the first aspect the compound of formula (I) is administered in an amount of 30 mg (BID), the dosing schedule is 3 days on/4 days off for three weeks, followed by a one week break without treatment.

In accordance with an embodiment of the first aspect the compound of formula (I) is administered in an amount of 30 mg (BID) and the dosing schedule is 4 days on/3 days off.

In accordance with an embodiment of the first aspect the compound of formula (I) is administered in an amount of 30 mg (BID) and the dosing schedule is 5 days on/2 days off.

In accordance with an embodiment of the first aspect the compound of formula (I) is administered in an amount of 30 mg (BID) and the dosing schedule is 6 days on/1 day off.

In accordance with an embodiment of the first aspect the compound of formula (I) is administered in an amount of 50 mg per day. The daily amount can be administered once daily (QD) or twice daily (BID). Preferably it is administered twice daily (BID), which means, that 25 mg are administered to the patient two times per day, for example, a first dose of 25 mg in the morning and a second dose of 25 mg in the evening Most preferably, the compound of formula (I) is administered twice daily (BID) every 12±1 hours.

In accordance with an embodiment of the first aspect the compound of formula (I) is administered in an amount of 25 mg (BID) and the dosing schedule is 3 days on/4 days off.

In accordance with an embodiment of the first aspect the compound of formula (I) is administered in an amount of 25 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 25 mg (BID), the dosing schedule is 3 days on/4 days off for two weeks, followed by a one week break without treatment.

In an embodiment of the first aspect the compound of formula (I) is administered in an amount of 25 mg (BID), the dosing schedule is 3 days on/4 days off for three weeks, followed by a one week break without treatment.

In accordance with an embodiment of the first aspect the compound of formula (I) is administered in an amount of 25 mg (BID) and the dosing schedule is 4 days on/3 days off.

In accordance with an embodiment of the first aspect the compound of formula (I) is administered in an amount of 25 mg (BID) and the dosing schedule is 5 days on/2 days off.

In accordance with an embodiment of the first aspect the compound of formula (I) is administered in an amount of 25 mg (BID) and the dosing schedule is 6 days on/1 day off.

In accordance with an embodiment of the first aspect the compound of formula (I) is administered in an amount of 40 mg per day. The daily amount can be administered once daily (QD) or twice daily (BID). Preferably it is administered twice daily (BID), which means, that 20 mg are administered to the patient two times per day, for example, a first dose of 20 mg in the morning and a second dose of 20 mg in the evening Most preferably, the compound of formula (I) is administered twice daily (BID) every 12±1 hours.

In accordance with an embodiment of the first aspect the compound of formula (I) is administered in an amount of 20 mg (BID) and the dosing schedule is 3 days on/4 days off.

In accordance with an embodiment of the first aspect the compound of formula (I) is administered in an amount of 20 mg (BID) and the dosing schedule is 3 days on/11 days off. In other words, in this embodiment the dosing schedule is 3 days on/4 days off for one week, followed by a one week break without treatment.

In accordance with an embodiment of the first aspect the compound of formula (I) is administered in an amount of 20 mg (BID), the dosing schedule is 3 days on/4 days off for two weeks, followed by a one week break without treatment.

In an embodiment of the first aspect the compound of formula (I) is administered in an amount of 20 mg (BID), the dosing schedule is 3 days on/4 days off for three weeks, followed by a one week break without treatment.

In accordance with an embodiment of the first aspect the compound of formula (I) is administered in an amount of 20 mg (BID) and the dosing schedule is 4 days on/3 days off.

In accordance with an embodiment of the first aspect the compound of formula (I) is administered in an amount of 20 mg (BID) and the dosing schedule is 5 days on/2 days off.

In accordance with an embodiment of the first aspect the compound of formula (I) is administered in an amount of 20 mg (BID) and the dosing schedule is 6 days on/1 day off.

In accordance with an embodiment of the first aspect the compound of formula (I) is administered in an amount of 20 mg (BID) and the dosing schedule is continuous. In other words, in this dosing schedule the 20 mg (BID) is administered every day (=7 days on/0 days off).

In accordance with an embodiment of the first aspect the compound of formula (I) is administered in an amount of 20 mg (BID), the dosing schedule is continuous for one week, followed by a one week break without treatment.

In accordance with an embodiment of the first aspect the compound of formula (I) is administered in an amount of 20 mg (BID), the dosing schedule is continuous for two weeks, followed by a one week break without treatment.

In accordance with an embodiment of the first aspect the compound of formula (I) is administered in an amount of 20 mg (BID), the dosing schedule is continuous for three weeks, followed by a one week break without treatment.

In accordance with an embodiment of the first aspect the compound of formula (I) is administered in an amount of 30 mg per day. The daily amount can be administered once daily (QD) or twice daily (BID). Preferably it is administered twice daily (BID), which means, that 15 mg are administered to the patient two times per day, for example, a first dose of 15 mg in the morning and a second dose of 15 mg in the evening Most preferably, the compound of formula (I) is administered twice daily (BID) every 12±1 hours.

In accordance with an embodiment of the first aspect the compound of formula (I) is administered in an amount of 15 mg (BID) and the dosing schedule is 3 days on/4 days off.

In accordance with an embodiment of the first aspect the compound of formula (I) is administered in an amount of 15 mg (BID) and the dosing schedule is 4 days on/3 days off.

In accordance with an embodiment of the first aspect the compound of formula (I) is administered in an amount of 15 mg (BID) and the dosing schedule is 5 days on/2 days off.

In accordance with an embodiment of the first aspect the compound of formula (I) is administered in an amount of 15 mg (BID) and the dosing schedule is 6 days on/1 day off.

In accordance with an embodiment of the first aspect the compound of formula (I) is administered in an amount of 15 mg (BID) and the dosing schedule is continuous. In other words, in this dosing schedule the 15 mg (BID) is administered every day (=7 days on/0 days off).

In accordance with an embodiment of the first aspect the compound of formula (I) is administered in an amount of 15 mg (BID), the dosing schedule is continuous for one week, followed by a one week break without treatment.

In accordance with an embodiment of the first aspect the compound of formula (I) is administered in an amount of 15 mg (BID), the dosing schedule is continuous for two weeks, followed by a one week break without treatment.

In accordance with an embodiment of the first aspect the compound of formula (I) is administered in an amount of 15 mg (BID), the dosing schedule is continuous for three weeks, followed by a one week break without treatment.

In accordance with an embodiment of the first aspect the compound of formula (I) is administered in an amount of 20 mg per day. The daily amount can be administered once daily (QD) or twice daily (BID). Preferably it is administered twice daily (BID), which means, that 10 mg are administered to the patient two times per day, for example, a first dose of 10 mg in the morning and a second dose of 10 mg in the evening Most preferably, the compound of formula (I) is administered twice daily (BID) every 12±1 hours.

In accordance with an embodiment of the first aspect the compound of formula (I) is administered in an amount of 10 mg (BID) and the dosing schedule is 5 days on/2 days off.

In accordance with an embodiment of the first aspect the compound of formula (I) is administered in an amount of 10 mg (BID) and the dosing schedule is 6 days on/1 day off.

In accordance with an embodiment of the first aspect the compound of formula (I) is administered in an amount of 10 mg (BID) and the dosing schedule is continuous. In other words, in this dosing schedule the 10 mg (BID) is administered every day (=7 days on/0 days off).

In accordance with an embodiment of the first aspect the compound of formula (I) is administered in an amount of 10 mg (BID), the dosing schedule is continuous for one week, followed by a one week break without treatment.

In accordance with an embodiment of the first aspect the compound of formula (I) is administered in an amount of 10 mg (BID), the dosing schedule is continuous for two weeks, followed by a one week break without treatment.

In accordance with an embodiment of the first aspect the compound of formula (I) is administered in an amount of 10 mg (BID), the dosing schedule is continuous for three weeks, followed by a one week break without treatment.

In accordance with an embodiment of the first aspect the compound of formula (I) is administered in an amount of 10 mg per day. The daily amount can be administered once daily (QD) or twice daily (BID). Preferably it is administered twice daily (BID), which means, that 5 mg are administered to the patient two times per day, for example, a first dose of 5 mg in the morning and a second dose of 5 mg in the evening Most preferably, the compound of formula (I) is administered twice daily (BID) every 12±1 hours.

In accordance with an embodiment of the first aspect the compound of formula (I) is administered in an amount of 5 mg (BID) and the dosing schedule is 6 days on/1 day off.

In accordance with an embodiment of the first aspect the compound of formula (I) is administered in an amount of 5 mg (BID) and the dosing schedule is continuous. In other words, in this dosing schedule the 5 mg (BID) is administered every day (=7 days on/0 days off).

In accordance with an embodiment of the first aspect the compound of formula (I) is administered in an amount of 5 mg (BID), the dosing schedule is continuous for one week, followed by a one week break without treatment.

In accordance with an embodiment of the first aspect the compound of formula (I) is administered in an amount of 5 mg (BID), the dosing schedule is continuous for two weeks, followed by a one week break without treatment.

In accordance with an embodiment of the first aspect the compound of formula (I) is administered in an amount of 5 mg (BID), the dosing schedule is continuous for three weeks, followed by a one week break without treatment.

In accordance with an embodiment of the first aspect the compound for use of the invention is characterized in that the compound of formula (I) is comprised in a pharmaceutical composition together with at least one pharmaceutically acceptable excipient.

The term “pharmaceutically acceptable excipient” is any excipient which is relatively non-toxic and innocuous to a patient at concentrations consistent with effective activity of the active ingredient, the compound of formula (I), so that any side effects ascribable to the excipient do not vitiate the beneficial effects of the active ingredient.

Pharmaceutically acceptable excipients in context with the present invention are for example fillers, disintegrants, lubricants, glidants, coating agents.

In accordance with an embodiment of the first aspect the compound for use of the invention is characterized in that the pharmaceutical composition comprises the compound of formula (I) in a portion of 25% by weight of the pharmaceutical composition or less, or in a portion of 20% by weight of the pharmaceutical composition or less, particularly in a portion of from 3 to 25%, preferably in a portion of from 4 to 22%, most preferred in a portion of from 5 to 20% by weight.

In accordance with an embodiment of the first aspect the compound for use of the invention is characterized in that the pharmaceutical composition comprises the compound of formula (I) in a portion of 25% by weight of the pharmaceutical composition or less, or in a portion of 20% by weight of the pharmaceutical composition or less, particularly in a portion of from 3 to 25%, preferably in a portion of from 4 to 22%, most preferred in a portion of from 5 to 20% by weight, and the pharmaceutical composition comprises a glidant. Preferably the pharmaceutical composition comprises a colloidal silicon dioxide (as a glidant).

In accordance with an embodiment of the first aspect the compound for use of the invention is characterized in that the pharmaceutical composition comprises the compound of formula (I) in a portion of 20% by weight of the pharmaceutical composition and the pharmaceutical composition comprises a glidant. Preferably the pharmaceutical composition comprises a colloidal silicon dioxide (as a glidant).

The term “glidant” refers to a pharmaceutically acceptable excipient that is added to the pharmaceutical composition to enhance the flow of the granulate by reducing interparticle friction. It includes pharmaceutically acceptable excipients such as colloidal silicon dioxide. Preferred glidant is colloidal silicon dioxide. The colloidal silicon dioxide can be characterized by a specific surface of from 50 to 600 m²·g⁻¹, preferably by a specific surface of from 100 to 300 m²·g⁻¹, most preferably of from 175 to 225 m²·g⁻¹.

In accordance with an embodiment of the first aspect the compound for use of the invention is characterized in that the pharmaceutical composition comprises lactose, preferably lactose monohydrate, most preferably spray-dried lactose monohydrate. Spray-dried lactose monohydrate is preferably characterized by a particle size distribution, in which 10% of the particles have a size of between 30 to 70 μm, 50% of the particles have a size of between 100 to 140 μm and 90% of the particles have a size of between 200 to 240 μm. The particle size distribution of spray-dried lactose monohydrate can be determined by laser diffraction.

In accordance with an embodiment of the first aspect the compound for use of the invention is characterized in that the pharmaceutical composition comprises the compound of formula (I) in an amount of from 5 mg to 80 mg, preferably in an amount of from 5 mg to 40 mg, more preferably in an amount of from 40 mg to 80 mg, and at least one pharmaceutically acceptable excipient.

In accordance with an embodiment of the first aspect the compound for use of the invention is characterized in that the pharmaceutical composition comprises the compound of formula (I) in an amount of 80 mg.

In accordance with a preferred embodiment of the first aspect the compound for use of the invention is characterized in that the pharmaceutical composition comprises the compound of formula (I) in an amount of 80 mg.

In accordance with an embodiment of the first aspect the compound for use of the invention is characterized in that the pharmaceutical composition comprises the compound of formula (I) in an amount of 70 mg.

In accordance with an embodiment of the first aspect the compound for use of the invention is characterized in that the pharmaceutical composition comprises the compound of formula (I) in an amount of 60 mg.

In accordance with a preferred embodiment of the first aspect the compound for use of the invention is characterized in that the pharmaceutical composition comprises the compound of formula (I) in an amount of 60 mg.

In accordance with an embodiment of the first aspect the compound for use of the invention is characterized in that the pharmaceutical composition comprises the compound of formula (I) in an amount of 50 mg.

In accordance with an embodiment of the first aspect the compound for use of the invention is characterized in that the pharmaceutical composition comprises the compound of formula (I) in an amount of 40 mg.

In accordance with a preferred embodiment of the first aspect the compound for use of the invention is characterized in that the pharmaceutical composition comprises the compound of formula (I) in an amount of 40 mg.

In accordance with an embodiment of the first aspect the compound for use of the invention is characterized in that the pharmaceutical composition comprises the compound of formula (I) in an amount of 30 mg.

In accordance with an embodiment of the first aspect the compound for use of the invention is characterized in that the pharmaceutical composition comprises the compound of formula (I) in an amount of 20 mg.

In accordance with an embodiment of the first aspect the compound for use of the invention is characterized in that the pharmaceutical composition comprises the compound of formula (I) in an amount of 10 mg.

In accordance with an embodiment of the first aspect the compound for use of the invention is characterized in that the pharmaceutical composition comprises the compound of formula (I) in an amount of 5 mg.

Pharmaceutical Composition

In accordance with a second aspect, the present invention relates to a pharmaceutical composition comprising the compound of formula (I) in an amount of from 5 mg to 80 mg, preferably in an amount of from 5 mg to 40 mg, more preferably in an amount of from 40 mg to 80 mg, and at least one pharmaceutically acceptable excipient.

In accordance with an embodiment of the second aspect, the pharmaceutical composition comprises the compound of formula (I) in an amount of 80 mg.

In accordance with a preferred embodiment of the second aspect, the pharmaceutical composition comprises the compound of formula (I) in an amount of 80 mg.

In accordance with an embodiment of the second aspect the pharmaceutical composition comprises the compound of formula (I) in an amount of 70 mg.

In accordance with an embodiment of the second aspect the pharmaceutical composition comprises the compound of formula (I) in an amount of 60 mg.

In accordance with a preferred embodiment of the second aspect, the pharmaceutical composition comprises the compound of formula (I) in an amount of 60 mg.

In accordance with an embodiment of the second aspect the pharmaceutical composition comprises the compound of formula (I) in an amount of 50 mg.

In accordance with a preferred embodiment of the second aspect the pharmaceutical composition comprises the compound of formula (I) in an amount of 40 mg.

In accordance with an embodiment of the second aspect the pharmaceutical composition comprises the compound of formula (I) in an amount of 30 mg.

In accordance with a preferred embodiment of the second aspect the pharmaceutical composition comprises the compound of formula (I) in an amount of 20 mg.

In accordance with a preferred embodiment of the second aspect the pharmaceutical composition comprises the compound of formula (I) in an amount of 10 mg.

In accordance with an embodiment of the second aspect the pharmaceutical composition comprises the compound of formula (I) in an amount of 5 mg.

In accordance with an embodiment of the second aspect the pharmaceutical composition of the invention comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight.

In accordance with an embodiment of the second aspect the pharmaceutical composition of the invention comprises the compound of formula (I) in a portion of from 3 to 10%, preferably in a portion of from 4 to 8%.

In accordance with an embodiment of the second aspect the pharmaceutical composition of the invention comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight, and the pharmaceutical composition comprises a glidant, such as colloidal silicon dioxide. Preferred glidant is colloidal silicon dioxide.

In accordance with an embodiment of the second aspect the pharmaceutical composition of the invention comprises the compound of formula (I) in a portion of from 3 to 10%, preferably in a portion of from 4 to 8%, and the pharmaceutical composition comprises a glidant, such as colloidal silicon dioxide.

Preferred glidant is colloidal silicon dioxide.

In accordance with an embodiment of the second aspect the pharmaceutical composition of the invention comprises lactose, particularly lactose monohydrate or lactose anhydrate, preferably spray-dried lactose monohydrate. A particularly preferable particle size distribution of spray-dried lactose monohydrate is described supra.

In accordance with an embodiment of the second aspect, the pharmaceutical composition of the invention comprises the compound of formula (I) in a portion of from 5 to 25%, one or more filler(s) in a portion of from 70 to 90%, one or more lubricant(s) in a portion of from 0.5 to 3%, one or more disintegrant(s) in a portion of from 2 to 10% and one or more glidant(s) in a portion of from 0.2 to 1% by weight of the pharmaceutical composition.

In accordance with an embodiment of the second aspect, the pharmaceutical composition of the invention comprises the compound of formula (I) in a portion of from 15 to 23%, one or more filler(s) in a portion of from 70 to 85%, one or more lubricant(s) in a portion of from 0.5 to 2%, one or more disintegrant(s) in a portion of from 2.5 to 7% and one or more glidant(s) in a portion of from 0.3 to 0.8% by weight of the pharmaceutical composition.

In accordance with an embodiment of the second aspect, the pharmaceutical composition of the invention comprises the compound of formula (I) in a portion of from 17 to 22%, one or more filler(s) in a portion of from 73 to 82%, one or more lubricant(s) in a portion of from 0.7 to 1.5%, one or more disintegrant(s) in a portion of from 3 to 6% and one or more glidant(s) in a portion of from 0.4 to 0.6% by weight of the pharmaceutical composition.

In accordance with an embodiment of the second aspect, the pharmaceutical composition of the invention comprises the compound of formula (I) in a portion of from 3 to 10%, one or more filler(s) in a portion of from 85 to 92%, one or more lubricant(s) in a portion of from 0.5 to 3%, one or more disintegrant(s) in a portion of from 2 to 10% and one or more glidant(s) in a portion of from 0.2 to 1% by weight of the pharmaceutical composition.

In accordance with an embodiment of the second aspect, the pharmaceutical composition of the invention comprises the compound of formula (I) in a portion of from 4 to 8%, one or more filler(s) in a portion of from 87 to 91%, one or more lubricant(s) in a portion of from 0.5 to 3%, one or more disintegrant(s) in a portion of from 2 to 10% and one or more glidant(s) in a portion of from 0.2 to 1% by weight of the pharmaceutical composition.

The term “filler” refers to a pharmaceutically acceptable excipient, which is added to the pharmaceutical composition to increase its bulk and/or weight. It includes pharmaceutically acceptable excipients such as microcrystalline cellulose and/or lactose.

Preferably, the filler is microcrystalline cellulose and/or lactose, particularly lactose monohydrate, preferably spray-dried lactose monohydrate, or the filler is a mixture of two or more fillers, such as a mixture of microcrystalline cellulose and lactose, particularly a mixture of microcrystalline cellulose and lactose monohydrate, preferably a mixture of microcrystalline cellulose and spray-dried lactose monohydrate. A particularly preferable particle size distribution of spray-dried lactose monohydrate is described supra.

In accordance with an embodiment of the second aspect, the pharmaceutical composition of the invention comprises one or more filler(s) in a portion of from 65 to 90% by weight of the pharmaceutical composition, preferably in a portion of from 70 to 85%, most preferably in a portion of from 73 to 77%.

In accordance with an embodiment of the second aspect, the pharmaceutical composition of the invention comprises one or more filler(s) in a portion of from 65 to 92% by weight of the pharmaceutical composition, preferably in a portion of from 70 to 92%, most preferably in a portion of from 73 to 91%.

The term “lubricant” refers to a pharmaceutically acceptable excipient, which is added to the pharmaceutical composition to reduce friction, heat, and wear when introduced between solid surfaces. It includes pharmaceutically acceptable excipients such as magnesium stearate. Preferably, the lubricant is magnesium stearate.

In accordance with an embodiment of the second aspect, the pharmaceutical composition of the invention comprises one or more lubricant(s) in a portion of from 0.5 to 3% by weight of the pharmaceutical composition, preferably in a portion of from 0.6 to 2%, most preferably in a portion of from 0.8 to 1.2%.

The term “disintegrant” refers to a pharmaceutically acceptable excipient, which is added to the pharmaceutical composition to cause its decomposition (disintegration) to support the release of the active ingredient from the pharmaceutical composition. It includes pharmaceutically acceptable excipients such as croscarmellose sodium, preference is given to croscarmellose sodium.

In accordance with an embodiment of the second aspect, the pharmaceutical composition of the invention comprises one or more disintegrant(s) in a portion of from 2 to 10% by weight of the pharmaceutical composition, preferably in a portion of from 2.5 to 6%, most preferably in a portion of from 3 to 5%.

The term “glidant” is defined supra.

In accordance with an embodiment of the second aspect, the pharmaceutical composition of the invention comprises one or more glidant(s) in a portion of from 0.2 to 1% by weight of the pharmaceutical composition, preferably in a portion of from 0.3 to 0.8%, most preferably in a portion of from 0.4 to 0.6%.

In accordance with an embodiment of the second aspect, the pharmaceutical composition of the invention comprises the compound of formula (I) in a portion of from 5 to 25%, microcrystalline cellulose (as a first filler) in a portion of from 40 to 51%, lactose monohydrate (as a second filler) in a portion of from 30 to 38%, magnesium stearate (as a lubricant) in a portion of from 0.5 to 2%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of from 10 to 24%, microcrystalline cellulose (as a first filler) in a portion of from 41 to 49%, lactose monohydrate (as a second filler) in a portion of from 30 to 36%, magnesium stearate (as a lubricant) in a portion of from 0.5 to 2%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of from 13 to 23%, microcrystalline cellulose (as a first filler) in a portion of from 41 to 47%, lactose monohydrate (as a second filler) in a portion of from 30 to 35%, magnesium stearate (as a lubricant) in a portion of from 0.5 to 2%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of from 16 to 22%, microcrystalline cellulose (as a first filler) in a portion of from 42 to 45%, lactose monohydrate (as a second filler) in a portion of from 31 to 33%, magnesium stearate (as a lubricant) in a portion of from 0.5 to 2%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of from 19 to 21%, microcrystalline cellulose (as a first filler) in a portion of from 42 to 43%, lactose monohydrate (as a second filler) in a portion of from 31 to 32%, magnesium stearate (as a lubricant) in a portion of from 0.5 to 2%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of 20%, microcrystalline cellulose (as a first filler) in a portion of from 42.8%, lactose monohydrate (as a second filler) in a portion of from 31.7%, magnesium stearate (as a lubricant) in a portion of 1%, croscarmellose sodium (as a disintegrant) in a portion of 4% and colloidal silicon dioxide (as a glidant) in a portion of 0.5% by weight of the pharmaceutical composition.

In accordance with an embodiment of the second aspect, the pharmaceutical composition of the invention comprises the compound of formula (I) in a portion of from 19 to 21%, microcrystalline cellulose (as a first filler) in a portion of from 42 to 43%, lactose monohydrate (as a second filler) in a portion of from 31 to 32%, magnesium stearate (as a lubricant) in a portion of from 1.1 to 1.5%, croscarmellose sodium (as a disintegrant) in a portion of from 2 to 8% and colloidal silicon dioxide (as a glidant) in a portion of from 0.2 to 0.8% by weight of the pharmaceutical composition.

In accordance with an embodiment of the second aspect, the pharmaceutical composition of the invention comprises the compound of formula (I) in a portion of 20%, microcrystalline cellulose (as a first filler) in a portion of from 42.7%, lactose monohydrate (as a second filler) in a portion of from 31.7%, magnesium stearate (as a lubricant) in a portion of 1.1%, croscarmellose sodium (as a disintegrant) in a portion of 4% and colloidal silicon dioxide (as a glidant) in a portion of 0.5% by weight of the pharmaceutical composition.

In accordance with an embodiment of the second aspect, the pharmaceutical composition of the invention comprises the compound of formula (I) in a portion of 20%, microcrystalline cellulose (as a first filler) in a portion of from 42.5%, lactose monohydrate (as a second filler) in a portion of from 31.7%, magnesium stearate (as a lubricant) in a portion of 1.3%, croscarmellose sodium (as a disintegrant) in a portion of 4% and colloidal silicon dioxide (as a glidant) in a portion of 0.5% by weight of the pharmaceutical composition.

In accordance with an embodiment of the second aspect, the pharmaceutical composition of the invention comprises the compound of formula (I) in a portion of 20%, microcrystalline cellulose (as a first filler) in a portion of from 42.3%, lactose monohydrate (as a second filler) in a portion of from 31.7%, magnesium stearate (as a lubricant) in a portion of 1.5%, croscarmellose sodium (as a disintegrant) in a portion of 4% and colloidal silicon dioxide (as a glidant) in a portion of 0.5% by weight of the pharmaceutical composition.

In accordance with an embodiment of the second aspect the pharmaceutical composition of the invention is used for oral administration.

In accordance with an embodiment of the second aspect the pharmaceutical composition of the invention is a solid oral dosage form.

The term “solid oral dosage form” includes granules, pellets, tablets, dragées, pills, melts, wafers or solid dispersions. Preferably the solid oral dosage form is selected from tablets (coated or uncoated), pellets and granules. Most preferably the solid oral dosage form is a tablet.

In accordance with an embodiment of the second aspect, the pharmaceutical composition of the invention can be an uncoated or a coated tablet.

Typical pharmaceutically acceptable tablet coating agents are hydroxyethyl cellulose, hydroxypropyl cellulose, methyl cellulose, hydroxypropyl methyl cellulose, sucrose, liquid glucose, ethyl cellulose, cellulose acetate phthalate, polyethylene glycol and shellac. The coating agents can be mixed with further applicable coating agents or commercially available ready-to-use coating mixtures can be used, such as for example Opadry™ 14F150002.

Preferably the tablet is coated with a mixture of hypromellose, polyethyleneglycol, titan dioxide, iron oxide, preferably red iron oxide, most preferred red Fe₂O₃, commercially available, for example, as Opadry 14F150002.

In accordance with an embodiment of the second aspect, the pharmaceutical composition of the invention is an immediate release tablet.

The pharmaceutical composition according to the invention shows good release properties. Furthermore preference is given to administration forms wherein the compound of formula (I) is released in a rapid manner also known as “immediate release” administration form. The term “immediate release” administration form refers to a release administration form having a Q-value (45 minutes) of 75%, wherein the Q-value is determined according to USP-release method Chapter <711> (USP 41-NF 36) with USP apparatus 2 (paddle apparatus).

In accordance with an embodiment of the second aspect, the pharmaceutical composition of the invention is the 10 mg tablet described in Example 1, Section 1.1.

In accordance with an embodiment of the second aspect, the pharmaceutical composition of the invention is the 20 mg tablet described in Example 1, Section 1.1.

In accordance with an embodiment of the second aspect, the pharmaceutical composition of the invention is the 40 mg tablet described in Example 1, Section 1.1.

In accordance with an embodiment of the second aspect, the pharmaceutical composition of the invention is the 40 mg tablet (Tablet D) described in Example 4.

In accordance with an embodiment of the second aspect, the pharmaceutical composition of the invention is the 40 mg tablet (Tablet E) described in Example 4.

In accordance with an embodiment of the second aspect, the pharmaceutical composition of the invention is the 40 mg tablet (Tablet F) described in Example 4.

In accordance with an embodiment of the second aspect, the pharmaceutical composition of the invention is the 60 mg tablet described in Example 3.

In accordance with an embodiment of the second aspect, the pharmaceutical composition of the invention is the 80 mg tablet described in Example 3.

Method for Treating

In accordance with a third aspect, the present invention relates to a method for treating a hyper-proliferative disease comprising administering the compound of formula (I) in an amount of from 10 mg to 160 mg per day to a patient, particularly in an amount of 60 mg to 160 mg per day, preferably in an amount of from 80 mg to 160 mg per day.

A patient, for the purpose of this invention, is a mammal, particularly a human, in need of treatment for the particular hyper-proliferative disease. The patient and/or the hyper-proliferative disease can be characterized by one or more defect(s) in the DNA damage response (DDR) machinery. Particularly, the patient and/or the hyper-proliferative disease can be characterized by one or more biomarker(s), which were described in International Patent Publication WO2018/153968, which is herein incorporated by reference.

Particularly, the patient and/or the hyper-proliferative disease can be characterized by one or more deleterious mutation(s) in one or more gene(s) and/or protein(s) further described in WO2018/153968. Preferably, the patient and/or the hyper-proliferative disease is characterized by one or more deleterious mutation(s) in ATM gene and/or BRCA1 gene and/or ATM protein and/or BRCA1 protein. For example, the deleterious mutation(s) in ATM gene and/or ATM protein may result in a loss of ATM protein and/or in a loss of ATM function. For example, the deleterious mutation(s) in BRCA1 gene and/or BRCA1 protein may result in a loss of BRCA1 protein and/or in a loss of BRCA1 function.

Further, the hyper-proliferative disease can be characterized by one or more deleterious mutation(s) in ATM gene and/or BRCA1 gene and/or BRCA2 gene and/or PALB2 gene and/or ARID1A gene and/or by a loss of ATM protein.

Preferably, the hyper-proliferative disease is characterized by one or more deleterious mutation(s) in ATM gene and/or BRCA1 gene and/or BRCA2 gene and/or by a loss of ATM protein.

Preferably, the hyper-proliferative disease is characterized by one or more deleterious mutation(s) in ATM gene.

Preferably, the hyper-proliferative disease is characterized by a loss of ATM protein.

Preferably, the hyper-proliferative disease is characterized by one or more deleterious mutation(s) in ATM gene and by a loss of ATM protein.

Preferably, the hyper-proliferative disease is characterized by one or more deleterious mutation(s) in BRCA1 gene.

Preferably, the hyper-proliferative disease is characterized by one or more deleterious mutation(s) in BRCA2 gene.

Further, the hyper-proliferative disease can be characterized by one or more deleterious mutation(s) in PALB2 gene.

Further, the hyper-proliferative disease can be characterized by one or more deleterious mutation(s) in ARID1A gene.

Methods for the determination of one or more deleterious mutation(s) in ATM or BRCA1 or BRCA2 or PALB2 or ARID1A gene and/or in ATM or BRCA1 or BRCA2 or PALB2 or ARID1A protein are known to the person skilled in the art and are described, for example, in WO2018/153968, which is herein incorporated by reference.

In accordance with an embodiment of the third aspect the compound of formula (I) is administered in in an amount of from 20 mg to 120 mg per day, or in an amount of from 60 mg to 160 mg per day, or in an amount of from 20 mg to 80 mg per day, preferably in an amount of from 80 mg to 160 mg per day.

In accordance with an embodiment of the third aspect the compound of formula (I) is administered in an amount of 160 mg per day. The daily amount can be administered once daily (QD) or twice daily (BID) as described supra.

In accordance with an embodiment of the third aspect the compound of formula (I) is administered in an amount of 80 mg (BID) by applying one or more of the dosing schedules for 80 mg (BID) treatment described supra in context with the first aspect of the invention (“Compound for use”).

In accordance with a preferred embodiment of the third aspect the compound of formula (I) is administered in an amount of 80 mg (BID) and the dosing schedule is 3 days on/11 days off.

In accordance with an embodiment of the third aspect the compound of formula (I) is administered in an amount of 140 mg per day. The daily amount can be administered once daily (QD) or twice daily (BID) as described supra.

In accordance with an embodiment of the third aspect the compound of formula (I) is administered in an amount of 70 mg (BID) by applying one or more of the dosing schedules for 70 mg (BID) treatment described supra in context with the first aspect of the invention (“Compound for use”).

In accordance with an embodiment of the third aspect the compound of formula (I) is administered in an amount of 120 mg per day. The daily amount can be administered once daily (QD) or twice daily (BID) as described supra.

In accordance with an embodiment of the third aspect the compound of formula (I) is administered in an amount of 60 mg (BID) by applying one or more of the dosing schedules for 60 mg (BID) treatment described supra in context with the first aspect of the invention (“Compound for use”).

In accordance with a preferred embodiment of the third aspect the compound of formula (I) is administered in an amount of 60 mg (BID) and the dosing schedule is 3 days on/11 days off.

In accordance with an embodiment of the third aspect the compound of formula (I) is administered in an amount of 110 mg per day. The daily amount can be administered once daily (QD) or twice daily (BID) as described supra.

In accordance with an embodiment of the third aspect the compound of formula (I) is administered in an amount of 55 mg (BID) by applying one or more of the dosing schedules for 55 mg (BID) treatment described supra in context with the first aspect of the invention (“Compound for use”).

In accordance with an embodiment of the third aspect the compound of formula (I) is administered in an amount of 100 mg per day. The daily amount can be administered once daily (QD) or twice daily (BID) as described supra.

In accordance with an embodiment of the third aspect the compound of formula (I) is administered in an amount of 50 mg (BID) by applying one or more of the dosing schedules for 50 mg (BID) treatment described supra in context with the first aspect of the invention (“Compound for use”).

In accordance with an embodiment of the third aspect the compound of formula (I) is administered in an amount of 90 mg per day. The daily amount can be administered once daily (QD) or twice daily (BID) as described supra.

In accordance with an embodiment of the third aspect the compound of formula (I) is administered in an amount of 45 mg (BID) by applying one or more of the dosing schedules for 45 mg (BID) treatment described supra in context with the first aspect of the invention (“Compound for use”).

In accordance with an embodiment of the third aspect the compound of formula (I) is administered in an amount of 80 mg per day. The daily amount can be administered once daily (QD) or twice daily (BID) as described supra.

In accordance with an embodiment of the third aspect the compound of formula (I) is administered in an amount of 40 mg (BID) by applying one or more of the dosing schedules for 40 mg (BID) treatment described supra in context with the first aspect of the invention (“Compound for use”).

In accordance with a preferred embodiment of the third aspect the compound of formula (I) is administered in an amount of 40 mg (BID) and the dosing schedule is 3 days on/4 days off.

In accordance with an embodiment of the third aspect the compound of formula (I) is administered in an amount of 70 mg per day. The daily amount can be administered once daily (QD) or twice daily (BID) as described supra.

In accordance with an embodiment of the third aspect the compound of formula (I) is administered in an amount of 35 mg (BID) by applying one or more of the dosing schedules for 35 mg (BID) treatment described supra in context with the first aspect of the invention (“Compound for use”).

In accordance with an embodiment of the third aspect the compound of formula (I) is administered in an amount of 60 mg per day. The daily amount can be administered once daily (QD) or twice daily (BID) as described supra.

In accordance with an embodiment of the third aspect the compound of formula (I) is administered in an amount of 30 mg (BID) by applying one or more of the dosing schedules for 30 mg (BID) treatment described supra in context with the first aspect of the invention (“Compound for use”).

In accordance with an embodiment of the third aspect the compound of formula (I) is administered in an amount of 50 mg per day. The daily amount can be administered once daily (QD) or twice daily (BID) as described supra.

In accordance with an embodiment of the third aspect the compound of formula (I) is administered in an amount of 25 mg (BID) by applying one or more of the dosing schedules for 25 mg (BID) treatment described supra in context with the first aspect of the invention (“Compound for use”).

In accordance with an embodiment of the third aspect the compound of formula (I) is administered in an amount of 40 mg per day. The daily amount can be administered once daily (QD) or twice daily (BID) as described supra.

In accordance with an embodiment of the third aspect the compound of formula (I) is administered in an amount of 20 mg (BID) by applying one or more of the dosing schedules for 20 mg (BID) treatment described supra in context with the first aspect of the invention (“Compound for use”).

In accordance with an embodiment of the third aspect the compound of formula (I) is administered in an amount of 30 mg per day. The daily amount can be administered once daily (QD) or twice daily (BID) as described supra.

In accordance with an embodiment of the third aspect the compound of formula (I) is administered in an amount of 15 mg (BID) by applying one or more of the dosing schedules for 15 mg (BID) treatment described supra in context with the first aspect of the invention (“Compound for use”).

In accordance with an embodiment of the third aspect the compound of formula (I) is administered in an amount of 20 mg per day. The daily amount can be administered once daily (QD) or twice daily (BID) as described supra.

In accordance with an embodiment of the third aspect the compound of formula (I) is administered in an amount of 10 mg (BID) by applying one or more of the dosing schedules for 10 mg (BID) treatment described supra in context with the first aspect of the invention (“Compound for use”).

In accordance with an embodiment of the third aspect the compound of formula (I) is administered in an amount of 10 mg per day. The daily amount can be administered once daily (QD) or twice daily (BID) as described supra.

In accordance with an embodiment of the third aspect the compound of formula (I) is administered in an amount of 5 mg (BID) by applying one or more of the dosing schedules for 5 mg (BID) treatment described supra in context with the first aspect of the invention (“Compound for use”).

In accordance with an embodiment of the third aspect the compound of formula (I) is comprised in a pharmaceutical composition together with at least one pharmaceutically acceptable excipient.

In accordance with an embodiment of the third aspect the compound of formula (I) is comprised in a pharmaceutical composition together with at least one pharmaceutically acceptable excipient, wherein the pharmaceutical composition comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight.

In accordance with an embodiment of the third aspect the compound of formula (I) is comprised in a pharmaceutical composition together with at least one pharmaceutically acceptable excipient, wherein the pharmaceutical composition comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight, and the pharmaceutical composition comprises a glidant. Preferably the glidant is colloidal silicon dioxide.

In accordance with an embodiment of the third aspect, the method for treating a hyper-proliferative disease is characterized in that the pharmaceutical composition comprises lactose, preferably lactose monohydrate, most preferably spray-dried lactose monohydrate.

In accordance with an embodiment of the third aspect, the method for treating a hyper-proliferative disease is characterized in that the pharmaceutical composition comprises the compound of formula (I) in an amount of from 5 mg to 80 mg, preferably in an amount of from 5 mg to 40 mg, more preferably in an amount of from 40 mg to 80 mg, and at least one pharmaceutically acceptable excipient.

In accordance with an embodiment of the third aspect, the method for treating a hyper-proliferative disease is characterized in that the pharmaceutical composition comprises the compound of formula (I) in an amount of 80 mg.

In accordance with a preferred embodiment of the third aspect, the method for treating a hyper-proliferative disease is characterized in that the pharmaceutical composition comprises the compound of formula (I) in an amount of 80 mg.

In accordance with an embodiment of the third aspect, the method for treating a hyper-proliferative disease is characterized in that the pharmaceutical composition comprises the compound of formula (I) in an amount of 70 mg.

In accordance with an embodiment of the third aspect, the method for treating a hyper-proliferative disease is characterized in that the pharmaceutical composition comprises the compound of formula (I) in an amount of 60 mg.

In accordance with a preferred embodiment of the third aspect, the method for treating a hyper-proliferative disease is characterized in that the pharmaceutical composition comprises the compound of formula (I) in an amount of 60 mg.

In accordance with an embodiment of the third aspect, the method for treating a hyper-proliferative disease is characterized in that the pharmaceutical composition comprises the compound of formula (I) in an amount of 50 mg.

In accordance with an embodiment of the third aspect, the method for treating a hyper-proliferative disease is characterized in that the pharmaceutical composition comprises the compound of formula (I) in an amount of 40 mg.

In accordance with a preferred embodiment of the third aspect, the method for treating a hyper-proliferative disease is characterized in that the pharmaceutical composition comprises the compound of formula (I) in an amount of 40 mg.

In accordance with an embodiment of the third aspect, the method for treating a hyper-proliferative disease is characterized in that the pharmaceutical composition comprises the compound of formula (I) in an amount of 30 mg.

In accordance with an embodiment of the third aspect, the method for treating a hyper-proliferative disease is characterized in that the pharmaceutical composition comprises the compound of formula (I) in an amount of 20 mg.

In accordance with an embodiment of the third aspect, the method for treating a hyper-proliferative disease is characterized in that the pharmaceutical composition comprises the compound of formula (I) in an amount of 10 mg.

In accordance with an embodiment of the third aspect, the method for treating a hyper-proliferative disease is characterized in that the pharmaceutical composition comprises the compound of formula (I) in an amount of 5 mg.

In accordance with a preferred embodiment of the third aspect the compound of formula (I) is comprised in a pharmaceutical composition according to the second aspect of the present invention, which is further described supra.

Process for Manufacturing the Pharmaceutical Composition

In accordance with a fourth aspect, the present invention relates to a process for manufacturing a pharmaceutical composition according to the second aspect of the invention, in which the compound of formula (I) is mixed with at least one pharmaceutically acceptable excipient.

In accordance with an embodiment of the fourth aspect of the invention the process for manufacturing a pharmaceutical composition according to the invention the compound of formula (I) is mixed with at least one pharmaceutically acceptable excipient by using a granulation method.

The term “granulation method” includes wet-granulation and dry-granulation. Wet-granulation methods include methods such as fluid bed granulation and high shear wet granulation. Dry-granulation methods include methods such as direct compression, slugging and roller compaction.

Preferably, the granulation method used in the process for manufacturing a pharmaceutical composition according to the invention is a dry-granulation method, most preferably it is a roller compaction method.

In accordance with an embodiment of the fourth aspect of the invention the process for manufacturing a pharmaceutical composition of the invention is characterized in that
a) the compound of formula (I) is granulated with at least one pharmaceutically acceptable excipient,
b) the granulate obtained by step a) is mixed with a lubricant and, optionally, with one or more further pharmaceutically acceptable excipient(s), and, optionally,
c) the product obtained by step b) is coated with one or more further pharmaceutically acceptable coating agent(s).

In accordance with an embodiment of the fourth aspect of the invention step a) of the process for manufacturing a pharmaceutical composition of the invention is characterized in that the compound of formula (I) is granulated with the filler, preferably microcrystalline cellulose and lactose monohydrate, the lubricant, preferably magnesium stearate, and the disintegrant, preferably croscarmellose sodium, preferably by a dry granulation method, most preferably by roller compaction.

In accordance with an embodiment of the fourth aspect of the invention step b) of the process for manufacturing a pharmaceutical composition of the invention is characterized in that the granulate obtained by step a) is mixed with a lubricant, preferably magnesium stearate, a glidant, preferably colloidal silicon dioxide, a filler, preferably microcrystalline cellulose, and with a disintegrant, preferably croscarmellose sodium. The mixing is performed by using a suitable device, such as for example a tumbler blender for a suitable time period.

In accordance with an embodiment of the fourth aspect of the invention step c) of the process for manufacturing a pharmaceutical composition of the invention the product of step b) is coated with one or more further pharmaceutically acceptable coating agent(s). Preference is given to pharmaceutically acceptable coating agent(s) selected from the group of plasticizer, film-forming agents and colorants. Optionally an anti-tacking agent or opacifier can be used. The plasticizer, preferably polyethylene glycol, the film-forming agent, preferably hypromellose, and the colorants, preferably ferric oxide and titanium dioxide, are combined with film-coating liquids, preferably (purified) water, to result in a homogeneous coating suspension which is brought up to, preferably sprayed on the product of step b), preferably on the tablets in a suitable coating device, such as for example a perforated drum coater. Other pigments or water soluble dyes or combinations thereof can be used to modify the colour of the coating.

In accordance with an embodiment of the fourth aspect of the invention after step b) of the process for manufacturing a pharmaceutical composition of the invention, optionally, there is a step called b 1), in which the mix obtained by step b) is subdivided into single units and further processed to the desired administration form, for example filling into sachets or capsules. Optionally one or more further pharmaceutically acceptable excipients are added. Preferably the mix is subdivided into single units and then compressed to tablets. The compression to tablets can be performed by using a tablet press, such as for example a standard rotary tablet press.

In accordance with an embodiment of the fourth aspect of the invention the process for manufacturing a pharmaceutical composition of the invention is characterized in that
a) the compound of formula (I) is mixed with one or more filler(s) and one or more disintegrant(s),
b) the mix obtained by step a) is mixed with one or more lubricant(s),
c) the mix obtained by step b) is roller-compacted to obtain ribbons,
d) the ribbons obtained by step c) are sieved to obtain a granulate,
e) the granulate obtained by step d) is mixed with one or more filler(s), one or more disintegrant(s) and one or more glidant(s),
f) the mix obtained by step e) is mixed with one or more lubricant(s),
g) the mix obtained by step f) is compressed to obtain a tablet, and, optionally,
h) the tablet obtained by step g) is coated with one or more further pharmaceutically acceptable coating agent(s).

Pharmaceutically acceptable coating agents(s), which can be optionally applied, are described above in context with the second aspect of the invention.

In accordance with an embodiment of the fourth aspect of the invention, the process for manufacturing a pharmaceutical composition of the invention is characterized in that the filler(s), the disintegrant(s), the lubricant(s), the glidant(s) of the pharmaceutical composition of the invention, particularly as described supra in context with the second aspect of the present invention are used. Preferably, the preferred fillers, the preferred disintegrant, the preferred lubricant and the preferred glidant defined supra are used.

In another aspect the present invention also concerns a process for manufacturing a pharmaceutical composition according to the invention, in which the compound of formula (I) is mixed with at least one pharmaceutically acceptable excipient and the resulting mixture is directly compressed to obtain a tablet. Optionally, the tablet can be coated with one or more further pharmaceutically acceptable excipients.

In another aspect the present invention also concerns a process for manufacturing a pharmaceutical composition according to the invention, in which the compound of formula (I) is mixed with at least one pharmaceutically acceptable excipient and the resulting mixture is directly compressed and filled into capsules or sachets.

Use of the Compound of Formula (I) for the Manufacture of a Medicament for the Treatment of a Hyper-Proliferative Disease

In accordance with a fifth aspect, the present invention relates to a use of the compound of formula (I) for the manufacture of a medicament for the treatment of a hyper-proliferative disease, characterized in that the compound of formula (I) is administered in an amount of from 10 mg to 160 mg per day to a patient.

In accordance with an embodiment of the fifth aspect the compound of formula (I) is administered in in an amount of from 20 mg to 120 mg per day, or in an amount of from 60 mg to 160 mg per day, or in an amount of from 20 mg to 80 mg per day, preferably in an amount of from 80 mg to 160 mg per day.

In accordance with an embodiment of the fifth aspect the compound of formula (I) is administered in an amount of 160 mg per day. The daily amount can be administered once daily (QD) or twice daily (BID) as described supra.

In accordance with an embodiment of the fifth aspect the compound of formula (I) is administered in an amount of 80 mg (BID) by applying one or more of the dosing schedules for 80 mg (BID) treatment described supra in context with the first aspect of the invention (“Compound for use”).

In accordance with an embodiment of the fifth aspect the compound of formula (I) is administered in an amount of 140 mg per day. The daily amount can be administered once daily (QD) or twice daily (BID) as described supra.

In accordance with an embodiment of the fifth aspect the compound of formula (I) is administered in an amount of 70 mg (BID) by applying one or more of the dosing schedules for 70 mg (BID) treatment described supra in context with the first aspect of the invention (“Compound for use”).

In accordance with an embodiment of the fifth aspect the compound of formula (I) is administered in an amount of 120 mg per day. The daily amount can be administered once daily (QD) or twice daily (BID) as described supra.

In accordance with an embodiment of the fifth aspect the compound of formula (I) is administered in an amount of 60 mg (BID) by applying one or more of the dosing schedules for 60 mg (BID) treatment described supra in context with the first aspect of the invention (“Compound for use”).

In accordance with an embodiment of the fifth aspect the compound of formula (I) is administered in an amount of 110 mg per day. The daily amount can be administered once daily (QD) or twice daily (BID) as described supra.

In accordance with an embodiment of the fifth aspect the compound of formula (I) is administered in an amount of 55 mg (BID) by applying one or more of the dosing schedules for 55 mg (BID) treatment described supra in context with the first aspect of the invention (“Compound for use”).

In accordance with an embodiment of the fifth aspect the compound of formula (I) is administered in an amount of 100 mg per day. The daily amount can be administered once daily (QD) or twice daily (BID) as described supra.

In accordance with an embodiment of the fifth aspect the compound of formula (I) is administered in an amount of 50 mg (BID) by applying one or more of the dosing schedules for 50 mg (BID) treatment described supra in context with the first aspect of the invention (“Compound for use”).

In accordance with an embodiment of the fifth aspect the compound of formula (I) is administered in an amount of 90 mg per day. The daily amount can be administered once daily (QD) or twice daily (BID) as described supra.

In accordance with an embodiment of the fifth aspect the compound of formula (I) is administered in an amount of 45 mg (BID) by applying one or more of the dosing schedules for 45 mg (BID) treatment described supra in context with the first aspect of the invention (“Compound for use”).

In accordance with an embodiment of the fifth aspect the compound of formula (I) is administered in an amount of 80 mg per day. The daily amount can be administered once daily (QD) or twice daily (BID) as described supra.

In accordance with an embodiment of the fifth aspect the compound of formula (I) is administered in an amount of 40 mg (BID) by applying one or more of the dosing schedules for 40 mg (BID) treatment described supra in context with the first aspect of the invention (“Compound for use”).

In accordance with an embodiment of the fifth aspect the compound of formula (I) is administered in an amount of 70 mg per day. The daily amount can be administered once daily (QD) or twice daily (BID) as described supra.

In accordance with an embodiment of the fifth aspect the compound of formula (I) is administered in an amount of 35 mg (BID) by applying one or more of the dosing schedules for 35 mg (BID) treatment described supra in context with the first aspect of the invention (“Compound for use”).

In accordance with an embodiment of the fifth aspect the compound of formula (I) is administered in an amount of 60 mg per day. The daily amount can be administered once daily (QD) or twice daily (BID) as described supra.

In accordance with an embodiment of the fifth aspect the compound of formula (I) is administered in an amount of 30 mg (BID) by applying one or more of the dosing schedules for 30 mg (BID) treatment described supra in context with the first aspect of the invention (“Compound for use”).

In accordance with an embodiment of the fifth aspect the compound of formula (I) is administered in an amount of 50 mg per day. The daily amount can be administered once daily (QD) or twice daily (BID) as described supra.

In accordance with an embodiment of the fifth aspect the compound of formula (I) is administered in an amount of 25 mg (BID) by applying one or more of the dosing schedules for 25 mg (BID) treatment described supra in context with the first aspect of the invention (“Compound for use”).

In accordance with an embodiment of the fifth aspect the compound of formula (I) is administered in an amount of 40 mg per day. The daily amount can be administered once daily (QD) or twice daily (BID) as described supra.

In accordance with an embodiment of the fifth aspect the compound of formula (I) is administered in an amount of 20 mg (BID) by applying one or more of the dosing schedules for 20 mg (BID) treatment described supra in context with the first aspect of the invention (“Compound for use”).

In accordance with an embodiment of the fifth aspect the compound of formula (I) is administered in an amount of 30 mg per day. The daily amount can be administered once daily (QD) or twice daily (BID) as described supra.

In accordance with an embodiment of the fifth aspect the compound of formula (I) is administered in an amount of 15 mg (BID) by applying one or more of the dosing schedules for 15 mg (BID) treatment described supra in context with the first aspect of the invention (“Compound for use”).

In accordance with an embodiment of the fifth aspect the compound of formula (I) is administered in an amount of 20 mg per day. The daily amount can be administered once daily (QD) or twice daily (BID) as described supra.

In accordance with an embodiment of the fifth aspect the compound of formula (I) is administered in an amount of 10 mg (BID) by applying one or more of the dosing schedules for 10 mg (BID) treatment described supra in context with the first aspect of the invention (“Compound for use”).

In accordance with an embodiment of the fifth aspect the compound of formula (I) is administered in an amount of 10 mg per day. The daily amount can be administered once daily (QD) or twice daily (BID) as described supra.

In accordance with an embodiment of the fifth aspect the compound of formula (I) is administered in an amount of 5 mg (BID) by applying one or more of the dosing schedules for 5 mg (BID) treatment described supra in context with the first aspect of the invention (“Compound for use”).

In accordance with an embodiment of the fifth aspect the compound of formula (I) is comprised in a pharmaceutical composition together with at least one pharmaceutically acceptable excipient.

In accordance with an embodiment of the fifth aspect the compound of formula (I) is comprised in a pharmaceutical composition together with at least one pharmaceutically acceptable excipient, wherein the pharmaceutical composition comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight.

In accordance with an embodiment of the fifth aspect the compound of formula (I) is comprised in a pharmaceutical composition together with at least one pharmaceutically acceptable excipient, wherein the pharmaceutical composition comprises the compound of formula (I) in a portion of 25% by weight of the pharmaceutical composition or less, or in a portion of 20% by weight of the pharmaceutical composition or less, particularly in a portion of from 15 to 25%, preferably in a portion of from 18 to 22%, most preferred in a portion of 20% by weight, and the pharmaceutical composition comprises a glidant. Preferably the glidant is colloidal silicon dioxide.

In accordance with an embodiment of the fifth aspect, the use of the compound of formula (I) for the manufacture of a medicament for the treatment of a hyper-proliferative disease is characterized in that the pharmaceutical composition comprises lactose, preferably lactose monohydrate, most preferably spray-dried lactose monohydrate.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of from 5 mg to 80 mg, preferably in an amount of from 5 mg to 40 mg, more preferably in an amount of from 40 mg to 80 mg, and at least one pharmaceutically acceptable excipient.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 80 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 70 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 60 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 50 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 40 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 30 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 20 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 10 mg.

In accordance with an embodiment of the fifth aspect, the use of the compound of formula (I) for the manufacture of a medicament for the treatment of a hyper-proliferative disease is characterized in that the pharmaceutical composition comprises the compound of formula (I) in an amount of 5 mg.

In accordance with a preferred embodiment of the fifth aspect, the use of the compound of formula (I) for the manufacture of a medicament for the treatment of a hyper-proliferative disease is characterized in that the compound of formula (I) is comprised in a pharmaceutical composition according to the second aspect of the present invention, which is further described supra.

### EXAMPLES

### Example 1

### Immediate Release Tablet Comprising BAY1895344 (20% Drug Load)

1.1 Composition of BAY 1895344 10 mg, 20 mg and 40 mg Coated Tablets (20% Drug Load)

| | Tablet A | Tablet B | Tablet C |
|---|---|---|---|
| | 10 mg | 20 mg | 40 mg |
| Composition | [mg] | [mg] | [mg] |
| Drug substance | | | |
| BAY 1895344 micronized | 10.00 | 20.00 | 40.00 |
| Tablet core | | | |
| Cellulose, microcrystalline | 21.405 | 42.81 | 85.62 |
| (Avicel ® PH102) | | | |
| Lactose monohydrate, spray- | 15.845 | 31.69 | 63.38 |
| dried (SuperTab ®11SD) | | | |
| Croscarmellose sodium | 2.000 | 4.00 | 8.00 |
| Magnesium stearate | 0.500 | 1.00 | 2.00 |
| Colloidal silicon dioxide | 0.250 | 0.50 | 1.00 |
| CAS numbers: 112945-52-5 | | | |
| and 7631-86-9 | | | |
| Weight (uncoated tablet) | 50.0 | 100.0 | 200.0 |

Film-coating (Opadry Dark Red 14F150002
ready to use commercial coating system)

| | | | |
|---|---|---|---|
| Hypromellose 15 cP | 1.5750 | 2.10 | 3.60 |
| Macrogol (Polyethylenglycol | 0.5250 | 0.70 | 1.20 |
| 3350) | | | |
| Iron oxide red | 0.3675 | 0.49 | 0.84 |
| Titanium dioxide | 0.1575 | 0.21 | 0.36 |
| Weight (film-coating) | 2.6250 | 3.50 | 6.00 |
| Weight (coated tablet) | 52.6 | 103.5 | 206.0 |

a) Micronization

BAY 1895344 was micronized in a jet mill after deagglomeration in an impeller sieve mill (mesh size 1.6 mm) in production scale of 200 mm diameter. The milling parameters are 3-6 kg/h feed rate and 3-6 bar milling pressure.

b) Dry Granulation and Blending

2 kg of BAY 1895344 micronized, 4.061 kg of cellulose microcrystalline, 3.169 kg of spray-dried lactose monohydrate and 0.2 kg of croscarmellose sodium were blended in suitable equipment. 0.07 kg of magnesium stearate was then added and further blended in suitable equipment. The blend was roller compacted using a Gerteis Mini-Pactor (Press force: 9.0 KN/cm; Gap width 2.0 mm; Press roller speed: 2.5 rpm; Granulator speed: 70 rpm). The ribbons produced from the roller compaction were granulated with a 20 mesh (840 micron) screen. Afterwards 0.22 kg of cellulose microcrystalline, 0.2 kg of croscarmellose sodium and 0.05 kg of colloidal silicon dioxide anhydrous were blended with the granule. Finally 0.03 kg of magnesium stearate was added to the final blend. Blending steps were performed in a suitable blender.

c) Tableting

The blend of step b) was compressed on a rotary tablet press into tablets containing 10 mg, 20 mg and 40 mg of BAY1895344.

d) Film Coating

The commercially available Opadry™ 14F150002 dark red was combined with purified water to result in a homogenous coating suspension which was sprayed on the tablets in a perforated drum coater.

The formulation of Example 1 has also been manufactured in different, i.e. larger scales. The ratio of ingredients and the operating principle of the equipment was the same.

1.2 Properties of the Tablets

TABLE 1
Study of release of compound of formula (I)
in % by total weight of the composition.

| | 15 min | 30 min | 45 min | 60 min |
|---|---|---|---|---|
| Tablet A | 78% | 87% | 91% | 92% |
| Tablet B | 79% | 92% | 96% | 98% |
| Tablet C | 72% | 88% | 92% | 95% |

Each value represents the mean of 6 single results. USP apparatus 2 (Paddle Apparatus), 900 ml citrate buffer pH 3.5, 50 rpm (Tablet A and B), 55 rpm (Tablet C).

Immediate Release Tablet Comprising BAY1895344 (33% Drug Load) 2.1 Composition of BAY 1895344 40 mg Coated Tablet (33% Drug Load)

| | Tablet 40 mg |
|---|---|
| Composition | [mg] |
| Drug substance | |
| BAY 1895344 micronized | 40.0 |
| Tablet core | |
| Cellulose, microcrystalline (Avicel ® PH102) | 42.9 |
| Lactose monohydrate, milled (not spray-dried) | 31.2 |
| Croscarmellose sodium | 4.8 |
| Magnesium stearate | 1.2 |
| Weight (uncoated tablet) | 120.0 |
| Hypromellose 15 cP | 2.2 |
| Macrogol (Polyethylenglycol 3350) | 0.7 |
| Iron oxide red | 0.5 |
| Titanium dioxide | 0.2 |
| Weight (film-coating) | 3.6 |
| Weight (coated tablet) | 123.6 |

a) Micronization

BAY1895344 was micronized in a jet mill after deagglomeration in an impeller sieve mill (mesh size 1.6 mm) in production scale of 200 mm diameter. The milling parameters were 3-6 kg/h feed rate and 3-6 bar milling pressure.

b) Dry Granulation and Blending

666 g of BAY1895344 micronized, 264 g of cellulose microcrystalline, 519 g of milled lactose monohydrate and 40 g of croscarmellose sodium were blended in suitable equipment. 14 g of magnesium stearate was then added and further blended in suitable equipment. The blend was roller compacted. The ribbons produced from the roller compaction were granulated with a mesh screen. Afterwards 450 g of cellulose microcrystalline and 40 g of croscarmellose sodium were blended with the granule. Finally 6 g of magnesium stearate was added to the final blend. Blending steps were performed in a suitable blender.

c) Tableting

The blend of step b) was compressed on a single-punch tablet press into tablets containing 40 mg of BAY1895344. Serious flow issues with the blend were observed during the compression. The blend was compacting in the hopper and therefore not flowing into the die of the rotary tablet press. Consequently a compression of this formulation was not possible.

d) Film Coating

As the tablet manufacturing was not successful this manufacturing step was not performed.

### Example 2

### First-In-Human Trial of the Oral ATR Inhibitor BAY 1895344 in Patients with Advanced Solid Tumors

Methods:

Patients (pts) with advanced metastatic solid tumors resistant or refractory to standard treatment, with and without DDR defects, were treated with BAY 1895344 BID, 3 days on/4 days off continuously on 3-week cycles (ClinicalTrials.gov Identifier: NCT03188965).

Results:

18 pts were enrolled in 6 cohorts (5 mg, 10 mg, 20 mg, 40 mg, 60 mg and 80 mg), including pts with colorectal (4), breast (3), prostate (2) and ovarian (2) cancers. Median prior lines of treatment was 5. No dose-limiting toxicities (DLTs) were reported in the 5-40 mg cohorts. There were 2/3 DLTs in the 80 mg cohort (Grade 4 [G4] neutropenia; G4 neutropenia and G4 thrombocytopenia) and 2/7 DLTs in the 60 mg cohort (G4 neutropenia; G2 fatigue). The 40 mg BID 3 on/4 off was defined as the maximum tolerated dose (MTD). Most common treatment emergent adverse events included anemia, neutropenia, nausea and fatigue. Pharmacokinetics appeared dose-proportional with pharmacodynamic analysis showing modulation of pH2AX and/or pKAP1 in paired tumor biopsies at exposures associated with preclinical anti-tumor activity. In 13 pts with and without DDR defects treated at dose levels ≥40 mg BID the objective response rate (ORR) was 30.7% including 2/2 pts in the 40 mg cohort (appendix and urothelial cancer), 1/8 pts at 60 mg (breast), and 1/3 pts at 80 mg (endometrial). Notably, these 4 responders had ATM protein loss of expression and/or ATM mutation with a median treatment duration of 347 days (range 293 d-364 d). A BRCA1-mutant, olaparib-resistant ovarian cancer pt (60 mg) had a CA125 response and SD>10 months. An additional 41 patients have been enrolled in ongoing expansion cohorts in cancers with DDR defects (prostate, breast, gynecological, colorectal) or ATM protein loss (all-comers) with responses observed.

Conclusions: The ATR inhibitor BAY 1895344 can be tolerated at biologically active doses with antitumor activity against cancers with certain DDR defects, including ATM protein loss.

### Example 3

### Immediate Release Tablet Comprising BAY1895344 (20% Drug Load)

Composition of BAY 1895344 60 mg and 80 mg Coated Tablets (20% Drug Load)

| | Tablet 60 mg | Tablet 80 mg |
|---|---|---|
| Composition | [mg] | [mg] |
| Drug substance | | |
| BAY 1895344 micronized | 60.00 | 80.00 |
| Tablet core | | |
| Cellulose, microcrystalline | 128.43 | 171.24 |
| (Avicel ® PH102) | | |
| Lactose monohydrate, spray-dried | 95.07 | 126.76 |
| (SuperTab ®11SD) | | |
| Croscarmellose sodium | 12.00 | 16.00 |
| Magnesium stearate | 3.00 | 4.00 |
| Colloidal silicon dioxide | 1.50 | 2.00 |
| CAS numbers: 112945-52-5 and | | |
| 7631-86-9 | | |
| Weight (uncoated tablet) | 300.0 | 400.0 |

Film-coating (Opadry Dark Red 14F150002
ready to use commercial coating system)

| | | |
|---|---|---|
| Hypromellose 15 cP | 5.40 | 7.20 |
| Macrogol (Polyethylenglycol 3350) | 1.80 | 2.40 |
| Iron oxide red | 1.26 | 1.68 |
| Titanium dioxide | 0.54 | 0.72 |
| Weight (film-coating) | 9.00 | 12.00 |
| Weight (coated tablet) | 309.0 | 412.0 |

a) Micronization

BAY 1895344 is micronized in a jet mill after deagglomeration in an impeller sieve mill (mesh size 1.6 mm) in production scale of 200 mm diameter. The milling parameters are 3-6 kg/h feed rate and 3-6 bar milling pressure.

b) Dry Granulation and Blending

2 kg of BAY 1895344 micronized, 4.061 kg of cellulose microcrystalline, 3.169 kg of spray-dried lactose monohydrate and 0.2 kg of croscarmellose sodium are blended in suitable equipment. 0.07 kg of magnesium stearate is then added and further blended in suitable equipment. The blend is roller compacted using a Gerteis Mini-Pactor (Press force: 9.0 KN/cm; Gap width 2.0 mm; Press roller speed: 2.5 rpm; Granulator speed: 70 rpm). The ribbons produced from the roller compaction are granulated with a 20 mesh (840 micron) screen. Afterwards 0.22 kg of cellulose microcrystalline, 0.2 kg of croscarmellose sodium and 0.05 kg of colloidal silicon dioxide anhydrous are blended with the granule. Finally 0.03 kg of magnesium stearate is added to the final blend. Blending steps are performed in a suitable blender.

c) Tableting

The blend of step b) is compressed on a rotary tablet press into tablets containing 60 mg and 80 mg of BAY1895344.

d) Film Coating

The commercially available Opadry™ 14F150002 dark red is combined with purified water to result in a homogenous coating suspension which is sprayed on the tablets in a perforated drum coater.

### Example 4

### Immediate Release Tablet Comprising BAY1895344 (20% Drug Load)

Composition of BAY 1895344 40 mg Coated Tablets (20% Drug Load) with Different Amounts of Magnesium Stearate.

| | Tablet D | Tablet E | Tablet F |
|---|---|---|---|
| | 40 mg | 40 mg | 40 mg |
| | Total amount | Total amount | Total amount |
| | of magnesium | of magnesium | of magnesium |
| | stearate | stearate | stearate |
| | 1.1% (w/w) | 1.3% (w/w) | 1.5% (w/w) |
| Composition | [mg] | [mg] | [mg] |
| Drug substance | | | |
| BAY 1895344 | 40.00 | 40.00 | 40.00 |
| micronized | | | |
| Tablet core | | | |
| Cellulose, | 85.42 | 85.02 | 84.62 |
| microcrystalline | | | |
| (Avicel ® PH102) | | | |
| Lactose monohydrate, | 63.38 | 63.38 | 63.38 |
| spray-dried | | | |
| (SuperTab ®11SD) | | | |
| Croscarmellose | 8.00 | 8.00 | 8.00 |
| sodium | | | |
| Magnesium stearate | 2.20 | 2.60 | 3.00 |
| Colloidal silicon | 1.00 | 1.00 | 1.00 |
| dioxide | | | |
| CAS numbers: | | | |
| 112945-52-5 | | | |
| and 7631-86-9 | | | |
| Weight (uncoated | 200.0 | 200.0 | 200.0 |
| tablet) | | | |

Film-coating (Opadry Dark Red 14F150002
ready to use commercial coating system)

| | | | |
|---|---|---|---|
| Hypromellose 15 cP | 3.60 | 3.60 | 3.60 |
| Macrogol (Poly- | 1.20 | 1.20 | 1.20 |
| ethylenglycol 3350) | | | |
| Iron oxide red | 0.84 | 0.84 | 0.84 |
| Titanium dioxide | 0.36 | 0.36 | 0.36 |
| Weight (film-coating) | 6.00 | 6.00 | 6.00 |
| Weight (coated tablet) | 206.0 | 206.0 | 206.0 |

a) Micronization

BAY 1895344 was micronized in a jet mill after deagglomeration in an impeller sieve mill (mesh size 1.6 mm) in production scale of 200 mm diameter. The milling parameters are 3-6 kg/h feed rate and 3-6 bar milling pressure.

b) Dry Granulation and Blending

2 kg of BAY 1895344 micronized, 4.061 kg of cellulose microcrystalline, 3.169 kg of spray-dried lactose monohydrate and 0.2 kg of croscarmellose sodium were blended in suitable equipment. 0.07 kg of magnesium stearate was then added and further blended in suitable equipment. The blend was roller compacted using a Gerteis Mini-Pactor (Press force: 9.0 KN/cm; Gap width 2.0 mm; Press roller speed: 2.5 rpm; Granulator speed: 70 rpm). The ribbons produced from the roller compaction were granulated with a 20 mesh (840 micron) screen. Afterwards 0.22 kg of cellulose microcrystalline, 0.2 kg of croscarmellose sodium and 0.05 kg of colloidal silicon dioxide anhydrous were blended with the granule. Finally 0.04 kg (Tablet D)/0.06 kg (Tablet E)/0.08 kg (Tablet F) of magnesium stearate was added to the final blend. Blending steps were performed in a suitable blender.

c) Tableting

The blend of step b) was compressed on a rotary tablet press into tablets containing 10 mg, 20 mg and 40 mg of BAY1895344.

d) Film Coating

The commercially available Opadry™ 14F150002 dark red was combined with purified water to result in a homogenous coating suspension which was sprayed on the tablets in a perforated drum coater.

Compared to the tablets of Example 1, the tablets of Example 4 were improved for an embossed tooling.

### Example 5

### Liquid Service Formulation Comprising BAY1895344 (1 mg/ml and 4 mg/ml)

Composition of BAY 1895344 1 mg/ml and 4 mg/ml Liquid Service Formulations:

| | Solution 1 mg/ml | Solution 4 mg/ml |
|---|---|---|
| Composition | [g] | [g] |
| Drug substance | | |
| BAY 1895344 micronized | 0.040 | 0.160 |
| Excipients | | |
| Citric acid anhydrous | 0.800 | 0.800 |
| Sucralose | 0.040 | 0.040 |
| 1N Sodium hydroxide | q.s. (pH-adjustment | q.s. (pH-adjustment |
| | to pH 1.9-2.7) | to pH 1.9-2.7) |
| Water for injections | 39.420 | 39.420 |

a) Micronization

BAY 1895344 was micronized in a jet mill after deagglomeration in an impeller sieve mill (mesh size 1.6 mm) in production scale of 200 mm diameter. The milling parameters are 3-6 kg/h feed rate and 3-6 bar milling pressure.

b) Manufacturing of the Solution

BAY 1895344 was suspended in a partial amount of water for injection under stirring (drug substance concentration). Citric acid anhydrous and sucralose were dissolved in the remaining amount of water for injections at room temperature (base solution).

The BAY 1895344 drug substance concentrate was added into the base solution and dissolved under stirring. pH-adjustment with 1 N sodium hydroxide/citric acid anhydrous was only necessary when pH value was not within the defined pH-range of 1.9-2.7. The solution was filtrated before filling into bottles.

### Example 6

### First-In-Human Trial of the Oral ATR Inhibitor BAY 1895344 in Patients with Advanced Solid Tumors—NCT03188965 Study Update

Efficacy:

The majority of patients treated were on the MTD dose and schedule (40 mg BID 3 days on/4 days off, n=134), n=20 patients were treated with different doses but same schedule (3 days on/4 days off), and n=8 patients so far at an alternate dose and schedule 60 mg BID and 80 mg BID 3 days on/11 days off (4 weeks per cycle).

At the beginning of the dose escalation phase (Part A) patients were treated with a liquid service formulation (LSF) as described in Example 5. At later stages of the dose escalation phase, patients were switched from LSF to tablets (see Example 1, section 1.1, tablets with 20% drug load). During expansion phase some patients were switched from LSF to tablets, but the majority was treated with tablets only.

At MTD Dose (40 mg BID) and Schedule (3 Days on/4 Days Off) n=134:

Some patients were pre-selected for DDR defects (in part B), and some were not (in part A). They were across multiple tumor indications, including ATM protein loss agnostic cohort (comprising castration-resistant prostate cancer, breast cancer, gastric cholangiocarcinoma, pancreatic, esophageal, colorectal cancer, squamous cell carcinoma of tongue and other cohorts with tumor types like ovarian, endometrial, cervical, breast, appendix, urothelial, castration-resistant prostate cancer, bladder, cholangiocarcinoma and colorectal cancer.

DDR defects were analyzed by using the FoundationONE® CDx from Foundation Medicine Inc., USA, for identifying deleterious mutations (in the following abbreviated with “mut”, e.g. “ATMmut”, “BRCA1mut”, “BRCA2mut” etc.) of one or more of the following genes: ATM, BRCA1, BRCA2, CHEK2, FANCA, MSH2, MRE11A, PALB2, RAD51, ATRX, CDK12, CHEK1, PARP1, POLD1, XRCC2, and/or ARID1A.

Patients/indications with ATM protein loss, identified by an IHC method, in the following, are also referred to as “ATMloss”.

Responses were defined as either complete response (CR), partial response (PR) or durable stable disease (SD), which means a stable disease of 4 months or more (=SD≥4 m).

There were 5 patients with PRs at the MTD dose level and schedule (40 mg BID 3 days on/4 days off), all with documented DDR defects (specifically 2 ATMloss, 1 ATMmut, 1 BRCA1mut, and 1 BRCA2mut), in indications including urothelial collecting duct carcinoma, appendix, ovarian, esophageal, and breast cancer.

There were 22 patients with a durable SD (SD≥4 m) distributed as follows: colorectal cancer (6 patients), ovarian (5), breast (4), endometrial (3), castration-resistant prostate cancer (2), gastric (1), and pancreatic cancer (1).

18 (81.8%) of them had DDR defect including 5 patients with ATMloss, 4 with ATMmut, and 1 with ATMloss and ATMmut, 5 with BRCA1mut (all of the aforementioned with or without additional DDR defects), 1 with a deleterious mutation of PALB2 (“PALB2mut”) but no ATMloss/mut, no BRCA1mut and no BRCA2mut, and lastly, 2 out of these 18 patients showed a deleterious mutation of ARID1A (“ARID1Amut”) but no concurrent ATMloss/mut, no BRCA1mut and no BRCA2mut.

3 out of the 22 patients did not have a DDR defect on retrospective testing, and for 1 out of 22 patients no data on DDR defects was available.

At Dose Levels Other than 40 mg BID but Same Schedule (3 Days on/4 Days Off) n=20:

Patients were not pre-selected for DDR defects (part A mainly) and were scattered across various tumor indications including colorectal cancer, breast, ovarian, endometrial, castration-resistant prostate cancer, pancreatic, lung, and hepatocellular carcinoma.

There were 5 responders (same definition as above). 2 of them (40%) had PR and were breast and endometrial cancers, both with ATMloss and ATMmut. The remaining 3 patients had durable SD≥4 m and were ovarian (BRCA1mut), hepatocellular carcinoma (no data on DDR defects available=N/A), and colorectal cancer (N/A).

Across all dose levels (5 mg BID-80 mg BID) tested on the 3 days on/4 days off schedule, there were 7 PRs spanning 6 indications: urothelial collecting duct carcinoma, appendix, ovarian, ATM protein loss esophageal (abbreviated as “esophageal ATMloss”), endometrial (1 patient each), and breast cancer (2 patients). Others have experienced durable stable disease (SD≥4 m) with the following indications: colorectal cancer (7) ovarian (6), breast (4), endometrial (3), castration-resistant prostate cancer (2), gastric (1), hepatocellular carcinoma (1), and pancreatic cancer (1). Patients on doses 5 mg BID and 10 mg BID, did not experience any type of response (no PR, and no SD≥4 m).

Biomarker

All 7 PRs mentioned above had DDR defects (2 ATMloss and ATMmut+2 ATMloss+1 ATMmut+1 BRCA1mut+1 BRCA2mut), whereas 19 out of 25 SD≥4 m had DDR defects (5 patients with ATMloss, 4 with ATMmut, and 1 with ATMloss and ATMmut, 6 with BRCA1mut, 1 with PALB2mut, 2 with ARID1Amut), 3 out of 25 were N/A, and 3 out of 25 patients showed no DDR defects.

15 patients out of 32 responders (includes PR and SD≥4 as described above) had ATMmut and/or ATMloss. 11 patients out of 32 responders neither had a loss of ATM protein nor a deleterious mutation of the ATM gene. 3 patients out of 32 responders had no DDR defect, and 3 out of 32 responders had an unknown mutational status (N/A).

At Alternate Schedule, Part A.1: 60 mg BID and 80 mg BID Each at 3 Days on/11 Days Off:

There are currently 2 dose levels in this ongoing escalation.

The first dose level is 60 mg BID 3 days on/11 days off, where 6 patients were treated, with cholangiocarcinoma (1 patient), castration-resistant prostate cancer (2), and colorectal cancer (3). 4 out of 6 patients have known DDR defects (3 patients with ATMloss, and 1 with ATMmut and ATMloss), but all patients are eligible per inclusion criteria of having ATM protein loss and/or ATMmut. So far, 1 cholangiocarcinoma responder with SD≥4 m was identified.

The second dose level is 80 mg BID 3 days on/11 days off, where 5 patients were treated so far. One patient experienced a stable disease with a reduction of the tumor size of −11% while on treatment for 56 days (first efficacy assessment timepoint), and ongoing.

There were no DLTs (DLT=dose limiting toxicity) observed until now at both dose levels (60 mg BID and 80 mg BID).

### CONCLUSION

Overall, cumulative non-clinical and clinical information indicates promising anti-cancer activity and manageable risk profile of BAY 1895344 supporting favorable benefit risk assessment of the compound in indications with unmet medical need.

1. A method for treatment of a hyper-proliferative disease, comprising administering to a patient in need thereof a compound of formula (I), wherein the compound of formula (I) is 2-[(3R)-3-methylmorpholin-4-yl]-4-(1-methyl-1H-pyrazol-5-yl)-8-(1H-pyrazol-5-yl)-1,7 naphthyridine 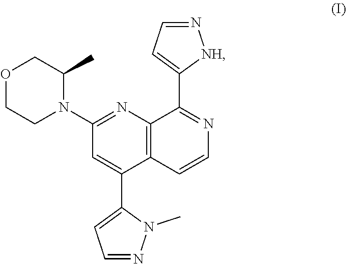 [SMILES: C[C@@H]1COCCN1c1cc(-c2ccnn2C)c2ccnc(-c3ccn[nH]3)c2n1]
in an amount of from 60 mg to 160 mg per day.

2. The method according to claim 1, wherein the compound of formula (I) is administered in an amount of from 80 mg to 160 mg per day.

3. The method according to claim 1, wherein the compound of formula (I) is administered in an amount of 80 mg per day.

4. The method according to claim 3, wherein the compound of formula (I) is administered in an amount of 40 mg (BID).

5. The method according to claim 4, wherein the dosing schedule is 3 days on/4 days off.

6. (canceled)

7. A pharmaceutical composition comprising a compound of formula (I), wherein the compound of formula (I) is 2-[(3R)-3-methylmorpholin-4-yl]-4-(1-methyl-1H-pyrazol-5-yl)-8-(1H-pyrazol-5-yl)-1,7 naphthyridine 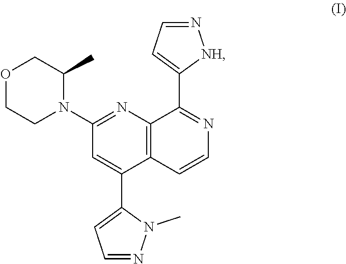 [SMILES: C[C@@H]1COCCN1c1cc(-c2ccnn2C)c2ccnc(-c3ccn[nH]3)c2n1]
in an amount of from 5 mg to 80 mg, and at least one pharmaceutically acceptable excipient.

8. The pharmaceutical composition of claim 7, comprising the compound of formula (I) in an amount of 40 mg.

9. The pharmaceutical composition of claim 7, comprising the compound of formula (I) in an amount of 20 mg.

10. The pharmaceutical composition of claim 7, comprising the compound of formula (I) in an amount of 10 mg.

11. The pharmaceutical composition of claim 7, comprising the compound of formula (I) in a portion of from 3 to 25% by weight of the pharmaceutical composition.

12. The pharmaceutical composition of claim 7, comprising a glidant.

13. The pharmaceutical composition of claim 12, wherein the glidant is colloidal silicon dioxide.

14. The pharmaceutical composition of claim 7, comprising spray-dried lactose.

15. The pharmaceutical composition of claim 7, comprising the compound of formula (I) in a portion of from 16 to 22%, microcrystalline cellulose in a portion of from 42 to 45%, lactose monohydrate in a portion of from 31 to 33%, magnesium stearate in a portion of from 0.5 to 2%, croscarmellose sodium in a portion of from 2 to 8% and colloidal silicon dioxide in a portion of from 0.2 to 0.8% by weight of the pharmaceutical composition.

16. A method for treatment of a hyper-proliferative disease, comprising administering to a patient in need thereof a pharmaceutical composition according to claim 7, wherein the compound of formula (I) is administered in an amount of from 60 mg to 160 mg per day.
